(19) United States
(12) Patent Application Publication
(10) Pub. No.: US 2024/0366171 A1
(43) Pub. Date: Nov. 7, 2024

(54) METHODS AND APPARATUSES FOR HYBRID CONE BEAM COMPUTED TOMOGRAPHIC AND INTRAORAL SCAN IMAGE MODELING

(71) Applicant: Align Technology, Inc., San Jose, CA (US)

(72) Inventors: Anna TYUTYUNNIK, Moscow (RU); Andrey VERIZHNIKOV, Moscow (RU); Samuel BLANCO, Santa Clara, CA (US); Evgenii Vladimirovich KARNYGIN, Moscow (RU); Maxim PETROV, Moscow (RU); Rene STERENTAL, Palo Alto, CA (US); Michael FLANAGAN, Redwood City, CA (US); Dmitry CHEKH, Moscow (RU); Savva KUZMIN, Moscow (RU); Mikhail KOLOBASHKIN, Moscow (RU); Andrey NEVOLIN, Moscow (RU)

(21) Appl. No.: 18/652,731

(22) Filed: May 1, 2024

Related U.S. Application Data
(60) Provisional application No. 63/499,478, filed on May 1, 2023.

(51) Int. Cl. A61B 6/00; A61B 6/40; A61B 6/51; G16H 30/20

(52) U.S. Cl. CPC A61B 6/5247; A61B 6/4085; A61B 6/512; G16H 30/20

(57) ABSTRACT

Methods and apparatuses for generating hybrid (e.g. fused) cone-beam computed tomography (CBCT) scan with an intraoral scan data. Also described herein are methods and apparatuses for more efficiently processing CBCT data. Also described herein are methods and apparatuses for using the hybrid CBCT and intraoral scan data to enhance dental and/or orthodontic treatment planning including interactively presenting the hybrid CBCT and intraoral scans and engaging with a user to generate one or more treatment plans and/or dental appliances for treating a patient according to a treatment plan that take into account the hybrid CBCT and intraoral scan.

### CLAIM OF PRIORITY

This patent application claims priority to U.S. provisional patent application No. 63/499,478, titled “METHODS AND APPARATUSES FOR HYBRID CONE BEAM COMPUTED TOMOGRAPHIC AND INTRAORAL SCAN IMAGE MODELING,” filed on May 1, 2023, and herein incorporated by reference in its entirety.

### BACKGROUND

Intraoral scanning is becoming increasingly standard for dental, and particularly orthodontic treatment planning. Unfortunately, most intraoral scanners only allow the visualization of the crowns and gingiva. Tooth roots and the surrounding bone (e.g., alveolar bone) are generally not visible and therefore cannot be directly taken into consideration when performing treatment planning. This can lead to problems once the treatment plan starts such as root collisions, fenestrations and unplanned and unpredictable movements of the teeth.

Although x-ray imaging, such as cone beam computed tomography (CBCT) may be available, CBCT scanning has proven difficult to integrate into most treatment planning process, particularly for orthodontic treatment planning, including the use of aligners (e.g., shell aligners). This may be due in part to the difference in resolution and scanning quality between CBCT and intraoral scans, as well as the relatively complexity in manipulating CBCT data and resulting data structures.

What is needed are methods and apparatuses (including software) that may integrate CBCT scanning into proven techniques, such as intraoral scanning, in order to better model a patient's dentition and improve patient outcomes.

### SUMMARY OF THE DISCLOSURE

Described herein are methods and apparatuses for generating hybrid (e.g. fused) cone-beam computed tomography (CBCT) scan with an intraoral scan data. Also described herein are methods and apparatuses for more efficiently processing CBCT data. Also described herein are methods and apparatuses for using the hybrid CBCT and intraoral scan data to enhance dental and/or orthodontic treatment planning. In particular, described herein are methods and apparatuses for interactively presenting the hybrid CBCT and intraoral scans and engaging with a user, such as a doctor, dentist, technician, etc., to generate one or more treatment plans and/or dental appliances for treating a patient according to a treatment plan that take into account the hybrid CBCT and intraoral scan.

The methods and apparatuses for forming the hybrid CBCT and intraoral scans described herein may overcome difficulties that have traditionally prevented the adoption and use of CBCT scanning in treatment planning and execution. These methods and apparatuses provide specific improvements over prior techniques and systems which were slow, difficult to implement and poorly suited for generating treatment planning and designing dental appliances which require a certain degree of precision to conform and apply force to a patient's teeth.

For example, the methods and apparatuses described herein may provide automatic (or semi-automatic) segmentation of CBCT data and fusion with intraoral scans. The resulting 3D digital models including the fused CBDT data and intraoral scan data may be interactively used to build treatment plans that take into consideration the actual tooth root geometry (e.g., long axis orientation), and that allow interactive visualization of the tooth roots and their movements in the treatment plan, including dynamically displaying predicted positions and orientations of the tooth roots, bone (e.g., alveolar bone) gingiva, etc., including prediction of problems such as collisions and fenestrations.

For example, described herein are methods of forming a hybrid (e.g., fused) digital model of a patient's dentition (e.g., upper and/or lower jaw, teeth crowns, roots, gingiva, bone, etc.). These methods and apparatuses (e.g., devices and systems, including in particular, software, hardware and/or firmware) provide a specific improvement over prior techniques and systems that attempted to used CBCT scanning and/or scans for orthodontic treatment planning. Such systems and techniques typically too long to upload and process, and may fail to allow combination with other scanning types (such as intraoral scans) or digital modeling based on other scanning types because the qualities of the CBCT scan may not be sufficient or sufficiently compatible with the other scanning types and/or models based on these other scanning types. In some of the methods and apparatuses described herein these problems may be overcome by preprocessing the CBCT scans locally, by a local processing agent, prior to transmission of the CBCT scan for combining with the intraoral scan (or other scan type) data. Local in the context of the CBCT scan may refer to local to the user (e.g., dentist, doctor, orthodontist, technician, etc.) and/or may refer to local to the site of storing and/or recording the CBCT scan. The CBCT scan may be fused with the intraoral scan at a remote site, by a remote processing agent, that is separate from the local processing agent. This may allow rapid and immediate feedback to the user, including processed quickly (providing rapid feedback), and rejecting scans that are not likely to work very early in the process.

For example, described herein are methods of fusing a cone-beam computed tomography (CBCT) scan with an intraoral scan. The method may include: receiving, in a remote processing agent, a processed CBCT scan file that has been processed by a local processing agent by: limiting the file size of a patient CBCT scan file to less than a maximum file size, and pre-segmenting by the remote processing agent to ensure that the processed CBCT scan file can be volumetrically segmented into individual teeth roots based on a scan quality of the CBCT scan and rejecting CBCT scan files that cannot be segmented; fusing the CBCT scan from the processed CBCT scan file with an intraoral scan of crowns of the patient's teeth to form a final model of the patient's teeth including the roots, wherein the processed CBCT scan has been volumetrically segmented and the intraoral scan has been surface segmented.

For example, described herein are methods of fusing a cone-beam computed tomography (CBCT) scan with an intraoral scan, the method comprising: receiving in a remote processing agent a processed CBCT scan file that has been processed by a local processing agent by: limiting the file size of a patient CBCT scan file to less than a maximum file size by truncating the file to remove one or more regions outside of any tooth roots, and by pre-segmenting to ensure that the processed CBCT scan file can be volumetrically segmented into individual teeth roots based on a scan quality of the CBCT scan and rejecting CBCT scan files that cannot be segmented; segmenting the processed CBCT scan file in the remote processing agent; fusing the segmented CBCT scan with a segmented intraoral scan of crowns of the patient's teeth to form a final model of the patient's teeth including the roots.

Receiving the processed CBCT scan file may include receiving the processed CBCT scan file that has been processed to limit the file size to less than the maximum file size by truncating the patient CBCT scan file (e.g., the “raw” CBCT scan file) to remove one or more regions outside of regions containing tooth roots. The maximum file size may be predetermined, or may be set, including adjusted based on connection speed. For example, the maximum file size may be 10 GB or less (e.g., 7.5 GB or less, 6 GB or less, 5 GB or less, 4 GB or less, 3 GB or less, 2 GB or less, etc.). The local processing agent may prevent a first user (e.g., an orthodontist requesting a treatment plan) from transmitting the CBCT scan file until the size is reduced. In general, the CBCT scan files described herein may be any appropriate format, including (but not limited to) a Digital Imaging and Communications in Medicine (DICOM) file.

In some examples receiving the processed CBCT scan file may include receiving the processed CBCT scan file that has been processed to limit the file size to less than the maximum file size by truncating the patient CBCT scan file to remove one or more layers of the patient CBCT scan file. For example, a DICOM file may be processed to manually or automatically remove one or more layers (“.dcm” files) from the top and/or bottom of the scan in cases where the scan covers vertically large areas outside of the teeth. Similarly, the local processing agent may adjust the other dimensions of the scan to remove regions that do not include the teeth (crowns and tooth roots and/or bone regions adjacent to the bone roots within a few mm).

Receiving the processed CBCT scan file may include receiving the processed CBCT scan file that has been processed to pre-segment the CBCT scan file based on the scan quality to reject CBCT scan files that cannot be segmented based on the scan quality that are blurry and/or below a minimum resolution threshold. The scan quality may generally include any indicator of visual quality and/or completeness of the scan. For example, the local agent may apply one or more thresholds for scan quality, including thresholds for blurriness, motion artifact, brightness (e.g., dynamic optical range), resolution, or the like. The local agent may determine the completeness of the CBCT scan, including confirming that the entire upper and/or lower jaw is present, the full extent of the teeth (roots, crowns, etc.) for all or a subset of the teeth, the bone, etc. The local processing agent may receive scan quality parameters from the remote processing agent. The local processing agent may also confirm that only a single CBCT scan is present.

The local processing agent may generally determine if the CBCT scan file is able to be segmented sufficiently for fusing with the second scan type (e.g. an intraoral scan). For example, the local processing agent may be determined based on the scan quality and scan completeness, as described above. In some examples the local processing agent may apply a trained neural network to determine if the CBCT scan file can be segmented, wherein the trained neural network is trained on database of CBCT scans having different scan qualities.

In many of the examples described herein segmentation may occur by the remote processing agent. However, in some examples, segmentation of the CBCT scan may be performed by the local processing agent and the fully segmented, or a partially segmented CBCT scan may be transmitted by the local processing agent to the remote processing agent as the processed CBCT scan. For example, receiving the processed CBCT scan file by the remote processing agent may include receiving the processed CBCT scan file that has been volumetrically segmenting by the local processing agent.

In any of these methods and apparatuses the CBCT scan file (e.g., the processed scan file) may be volumetrically segmented. For example, these methods may include volumetrically segmenting the received processed CBCT scan file.

The second scan to be fused with the CBCT scan may also be segmented. For example, the intraoral scan may be segmented. In some examples, the method may include segmenting the intraoral scan, e.g., by the remote processing agent. The CBCT scan may be segmented differently than the secondary scan (e.g., intraoral scan) for example, the CBCT scan may be volumetrically segmented while the intraoral scan may be segmented as a surface.

The processed, and in some examples segmented, CBCT scan may be fused with the intraoral scan. Fusing may include matching crown regions of the segmented intraoral scan with crown regions of the segmented CBCT scan and replacing the crown regions of the segmented CBCT scans with the crown regions of the segmented intraoral scanner. In some examples fusing may include modifying the crown regions of the segmented intraoral scan based on the crown regions of the segmented CBCT scan and using the modified crown regions. In some examples, fusing may include matching to confirm which portion of the segmented CBCT scan with the segmented intraoral scan. The crown regions of the intraoral and/or CBCT scan may be scaled and/or moved (e.g., rotated) relative to each other to align and match. In some examples individual or sub-sets of segmented crown region of the intraoral scan and/or teeth of the CBCT scan may be manipulated relative to each other as part of the fusion process. For example the crown regions of individual teeth of the intraoral scan may be individually manipulated for matching and fusion with the CBCT scan. The dimensions of the crown regions of the CBCT scans may be matched to the dimensions of the intraoral scan crowns.

In any of these examples fusing may include verifying the fusion based on degree of match between the crown regions of the segmented intraoral scan with crown regions of the segmented CBCT scan. For example, the methods and apparatuses described herein may apply a threshold to confirm that the fusion is adequate based on the degree of match.

The resulting final model of the patient's teeth may therefore include the roots and crowns for each tooth, with the CBCT scan improved by the intraoral scan of the crowns. The final model may include, or may be used to generate, a determination of the long axis of each tooth, taken from the root axis of each tooth in the CBCT scan. The resulting long axis may be used in treatment planning and/or predicting movements, collisions, fenestrations, etc.

Any of these methods and apparatuses (e.g., systems) may include a user interface. The user interface may be made particularly effective by the final model of the patient's teeth, including the fused (hybrid) CBCT/intraoral scan. For example, any of these methods may include displaying the final model of the patient's teeth in a user interface configured to allow a user to interactively display subsets of segmented regions of the final model. For example, allowing the user to interactively display subsets of segmented regions may include receiving user commands to show one or more of: unerupted teeth, roots only, surrounding semitransparent bone, and crowns only. The user may interactively alter the portions of the final model of the patient's teeth to show all or some of the segments (regions). The fused CBCT crowns, derived from the intraoral scan, may provide a very high fidelity representation of the tooth crown surface(s), while the CBCT roots and other segmented regions (e.g. bone) may provide a larger context.

Any of the methods described herein, including the methods of fusing a cone-beam computed tomography (CBCT) scan with an intraoral scan may be performed by an apparatus, including a system, device etc., including software. For example, described herein are non-transitory computing device readable medium having instructions stored thereon that are executable to perform any of these methods. For example, a methods as described herein may include a non-transitory computing device readable medium having instructions stored thereon that are executable by a processor of a remote processing agent to cause the remote processing agent to perform a method comprising: receiving a processed CBCT scan file that has been processed by a local processing agent by: limiting the file size of a patient CBCT scan file to less than a maximum file size, and pre-segmenting by the remote processing agent to ensure that the processed CBCT scan file can be volumetrically segmented into individual teeth roots based on a scan quality of the CBCT scan and rejecting CBCT scan files that cannot be segmented; fusing the CBCT scan from the processed CBCT scan file with an intraoral scan of crowns of the patient's teeth to form a final model of the patient's teeth including the roots, wherein the processed CBCT scan has been volumetrically segmented and the intraoral scan has been surface segmented.

For example, a non-transitory computing device readable medium having instructions stored thereon that are executable by a processor of a remote processing agent may cause the remote processing agent to perform a method comprising: receiving in a remote processing agent a processed CBCT scan file that has been processed by a local processing agent by: limiting the file size of a patient CBCT scan file to less than a maximum file size by truncating the file to remove one or more regions outside of any tooth roots, and by pre-segmenting to ensure that the processed CBCT scan file can be volumetrically segmented into individual teeth roots based on a scan quality of the CBCT scan and rejecting CBCT scan files that cannot be segmented; segmenting the processed CBCT scan file in the remote processing agent; fusing the segmented CBCT scan with a segmented intraoral scan of crowns of the patient's teeth to form a final model of the patient's teeth including the roots.

Also described herein are methods and apparatuses that may be performed by the local processing agent. For example a method of processing a cone-beam computed tomography (CBCT) scan may include: accessing a patient CBCT scan file using a local software processing agent; limiting the file size of the CBCT scan file to less than a maximum file size; pre-segmenting the CBT scan, by the remote processing agent, to ensure that the processed CBCT scan file can be volumetrically segmented into individual teeth roots based on a scan quality of the CBCT scan and rejecting CBCT scan files that cannot be segmented; and uploading the processed CBCT scan file to a remote site for fusing with an intraoral scan from the patient.

Any of these methods may be combined in whole or in part (e.g., the methods may include methods performed by both the local and remote processing agents).

As mentioned above, limiting the file size of the CBCT scan file to less than a maximum file size may include truncating the CBCT scan file to remove one or more regions outside of regions containing tooth roots. In some examples limiting the file size comprises truncating the patient CBCT scan file to remove one or more layers of the patient CBCT scan file.

Pre-segmenting the CBCT scan file based on the scan quality may include rejecting CBCT scan files that cannot be segmented based on the scan quality that are blurry and/or below a minimum resolution threshold. For example, pre-segmenting the CBCT scan file based on the scan quality comprises applying a trained neural network to determine if the CBCT scan file can be segmented, wherein the trained neural network is trained on database of CBCT scans having different scan qualities.

Any of these methods may include volumetrically segmenting the processed CBCT scan file by the local processing agent, e.g., prior to transmitting the processed CBCT scan. Alternatively, the processed CBCT scan file may be segmented by a remote processing agent. As mentioned, the CBCT scan file may be any appropriate file type, including a Digital Imaging and Communications in Medicine (DICOM) file.

Any of these methods and apparatuses described herein may include displaying one or more messages on the scan quality of the CBCT scan file to a user before uploading the processed CBCT scan file.

For example, a method of processing a cone-beam computed tomography (CBCT) scan may include: accessing a patient CBCT scan file using a local software processing agent; limiting the file size of the CBCT scan file to less than a maximum file size by truncating the CBCT scan file to remove one or more regions outside of regions containing tooth roots; pre-segmenting the CBT scan, by the remote processing agent, to ensure that the processed CBCT scan file can be volumetrically segmented into individual teeth roots based on a scan quality of the CBCT scan and rejecting CBCT scan files that cannot be segmented, wherein scan quality comprises one or more of: blurriness and resolution; and uploading the processed CBCT scan file to a remote site for fusing with an intraoral scan from the patient.

Also described herein are one or more local processing agents that may be part of a system including a remote processing agent. The local processing agent may perform any of the methods described herein for pre-processing a CBCT scan. For example, a local processing agent for processing a cone-beam computed tomography (CBCT) scan may include a non-transitory computing device readable medium having instructions stored thereon that are executable by a processor of a remote processing agent to cause the remote processing agent to perform a method comprising: accessing a patient CBCT scan file using a local software processing agent; limiting the file size of the CBCT scan file to less than a maximum file size; pre-segmenting the CBT scan, by the remote processing agent, to ensure that the processed CBCT scan file can be volumetrically segmented into individual teeth roots based on a scan quality of the CBCT scan and rejecting CBCT scan files that cannot be segmented; and uploading the processed CBCT scan file to a remote site for fusing with an intraoral scan from the patient.

In some examples a local processing agent for processing a cone-beam computed tomography (CBCT) scan may include a non-transitory computing device readable medium having instructions stored thereon that are executable by a processor of a remote processing agent to cause the remote processing agent to perform a method comprising: accessing a patient CBCT scan file using a local software processing agent; limiting the file size of the CBCT scan file to less than a maximum file size by truncating the CBCT scan file to remove one or more regions outside of regions containing tooth roots; pre-segmenting the CBT scan, by the remote processing agent, to ensure that the processed CBCT scan file can be volumetrically segmented into individual teeth roots based on a scan quality of the CBCT scan and rejecting CBCT scan files that cannot be segmented, wherein scan quality comprises one or more of: blurriness and resolution; and uploading the processed CBCT scan file to a remote site for fusing with an intraoral scan from the patient.

In general, the fused CBCT scan and intraoral scan (e.g., the final model of the patient's teeth including the roots, which is segmented) may be used for treatment planning, and in particular, for interactive treatment planning to allow a user to display and manipulate digital representations of the patient's teeth to more quickly and accurately predict tooth movement and potential defect, such as fenestrations, that may result from one or more proposed movements as part of a treatment plan. Thus, also described herein are methods and apparatuses for interactively displaying and predicting tooth movements and potential defects of tooth movement, particularly defects involving interactions with the surrounding bone (e.g., alveolar bone) and gingiva. The methods and apparatuses described herein represent a significant and specific improvement over prior art systems, as these methods (and systems for performing them) may predict fenestrations and allow the user to adjust the movement of the teeth in a treatment plan and see changes in predicted fenestrations in response to the changes in the treatment plan. This may be done in real time. This improvement is possible in part because of the use of the segmented fused CBCT and intraoral scan that includes the roots and bone (from the CBCT scan) and the crowns (from the intraoral scan) as well as the properties of the user interface.

For example, described herein are method and apparatuses for predicting and visualizing possible fenestration resulting from one or more stages of a proposed treatment plan; these methods and apparatuses may be configured to allow a user to interactively modify the treatment plan to adjust the proposed treatment plan and see the effect on the relationship between the teeth, the teeth and the alveolar bone, and/or the gingiva. Any of these methods and apparatuses may be particularly useful for predicting fenestrations.

For example, a method may include receiving a digital three-dimensional (3D) model of a patient's teeth, wherein the 3D model of the patient's teeth comprises a fusion of a CBCT scan of the patient's jaw and an intraoral scan of the patient's crown regions of the patient's teeth; simulating, using the 3D model of the patient's teeth, the movement of teeth roots relative to the patient's alveolar bone for one or more steps of a treatment plan for orthodontically moving the patient's teeth; identifying the formation of one or more fenestrations of the teeth roots from the simulation for the one or more steps of the treatment plan; and displaying the 3D model of the patient's teeth for the one or more steps of the treatment plan showing the identified one or more fenestrations.

Any of these methods may include receiving one or more modification to the treatment plan from the user; and revising the simulation of the movement of the teeth roots relative to the patient's alveolar bone and displaying the revised 3D model of the patient teeth showing the identified one or more fenestrations.

The method may include receiving a command from the user to display fenestrations. For example, receiving the command may include interactively, based on user input, switching between the stages of the treatment plan and displaying the 3D model of the patient's teeth showing the identified one or more fenestrations.

User input may be received via a user interface that may include displaying one or more images of the patient's teeth, including selectably displaying one or more of: crowns, roots, gingiva, alveolar bone.

Any of these methods may include identifying the formation of one or more dehiscence of the teeth roots from the simulation for the one or more steps of the treatment plan and displaying the 3D model of the patient's teeth for the one or more steps of the treatment plan showing the identified one or more dehiscence. Alternatively or additionally, any of these methods may include identifying the formation of one or more protrusions of the teeth roots from the alveolar bone (e.g., into the sinus cavity) in the simulation for the one or more steps of the treatment plan and displaying the 3D model of the patient's teeth for the one or more steps of the treatment plan showing the identified one or more protrusions. Simulating may include determining a long axis of each tooth using a long axis of the root portion of each tooth from the 3D model and modeling the movement of the root based on force applied to the crown of the tooth and the relatively positions of adjacent teeth, including root portions of the adjacent teeth.

For example, a method may include: receiving a digital three-dimensional (3D) model of a patient's teeth, wherein the 3D model of the patient's teeth comprises a fusion of a CBCT scan of the patient's jaw and an intraoral scan of the patient's crown regions of the patient's teeth; simulating, using the 3D model of the patient's teeth, the movement of teeth roots relative to the patient's alveolar bone for one or more steps of a treatment plan for orthodontically moving the patient's teeth; identifying the formation of one or more fenestrations of the teeth roots from the simulation for the one or more steps of the treatment plan; receiving a command from the user to display fenestrations; displaying the 3D model of the patient's teeth for the one or more steps of the treatment plan showing the identified one or more fenestrations; receiving one or more modification to the treatment plan from the user; and revising the simulation of the movement of the teeth roots relative to the patient's alveolar bone and displaying the revised 3D model of the patient teeth showing the identified one or more fenestrations.

In some examples a method may include: receiving a digital three-dimensional (3D) model of a patient's teeth, wherein the 3D model of the patient's teeth comprises a fusion of a CBCT scan of the patient's jaw and an intraoral scan of the patient's crown regions of the patient's teeth; simulating, using the 3D model of the patient's teeth, the movement of teeth roots relative to the patient's alveolar bone for one or more steps of a treatment plan for orthodontically moving the patient's teeth; identifying the formation of one or more alveolar bone defects comprising one or more of: fenestrations of the teeth roots from the simulation for the one or more steps of the treatment plan, dehiscence of the teeth roots from the simulation for the one or more steps of the treatment plan, and one or more protrusions of the teeth roots from the alveolar bone in the simulation for the one or more steps of the treatment plan and displaying the 3D model of the patient's teeth for the one or more steps of the treatment plan showing the identified one or more protrusions; and displaying the 3D model of the patient's teeth for the one or more steps of the treatment plan showing the identified one or more alveolar bone defects.

Thus, the methods and apparatuses described herein may predict the occurrence of one or more of: fenestrations, protrusions and dehiscence that may arise in treatment planning. Accurate predictions of fenestrations, protrusions and dehiscence may be possible by the use of the fused CBCT scan/intraoral scan digital models as described. In some cases the prediction of fenestrations, protrusions and dehiscence may be performed automatically for one or more stages of a proposed treatment plan. In some cases the method and/or apparatus may predict the likelihood of one or more of fenestrations, protrusions and dehiscence in a proposed treatment plan using the fused CBCT scan/intraoral scan digital model automatically for one or more stages and may, if the likelihood of the fenestrations, protrusions and/or dehiscence exceeds a threshold (e.g., a threshold for fenestrations, a threshold for protrusions, a threshold for dehiscence), the method and/or apparatus may display highlight and/or display the likely fenestrations, protrusions and/or dehiscence. Thus, in any of these methods and apparatuses, when planning orthodontic treatment, the user (e.g., doctor, dentist, orthodontist, etc.) may benefit from the use of the integration of the intraoral scan data on tooth crowns with the extended CBCT data, and in particular with the ability of the system to quickly and effectively form the fused and segmented digital scan of the patient's oral cavity including the CBCT scan data and the intraoral scan data. The resulting fused CBCT/intraoral scan data may be integrated into a treatment plan in the form of 3D surfaces of the teeth roots and the outer surface of the bone. This data can be used to detect possible problems caused by the proposed treatment or changes to the proposed treatment, including protrusions of a tooth or teeth into the nasal sinus and fenestration of the tooth into the jaw bone. These method and apparatuses may highlight parts of the surfaces of the teeth, roots, or bone that may be susceptible to fenestrations, protrusions and/or dehiscence, and generally to features that may lead to unpredictable movements of the teeth. Incorporating these methods and apparatuses into the treatment planning process, and in particular allowing visualization of the crowns, roots and alveolar bone may help avoid potential problems, and may significantly reduce the risk of unpredictable tooth movements including root collisions and fenestrations.

In general, these methods may be used to automatically segment CBCT data and fusion with intraoral scans, and building of treatment plans that take into consideration real root geometry (including long axis orientation).

As mentioned, these methods and apparatuses may include user interfaces that allow the user to visualize real roots and their movements in the treatment plan with various options, including displaying just the teeth with their actual (real) roots (taken from the CBCT scan data), displaying the teeth with their actual (real) roots with semitransparent bone, displaying the teeth with their actual (real) roots with opaque bone, displaying one or more reference static bone: Maxilla, Mandibula and parts of adjacent bones, and visualizing root collisions and possible virtual root fenestrations.

The user interface may be configured to allow the user to display and interact with the resulting 3D model of the patient's upper and/or lower jaw in a manner similar to how a traditional DICOM data file may be visualized (e.g., following CBCT scanning). These methods and apparatuses may be configured to allow the user to visualize unerupted, supernumerary and impacted teeth in a treatment plan, including the selection for display or hiding one or more of unerupted, supernumerary and impacted teeth. These methods and apparatuses may enhance the ability of the treatment plan to achieve root parallelism as part of the treatment plan. Any of these methods and apparatuses may be used for planning dental implants instead of or in addition to treatment planning for orthodontic alignment.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIGS. 1A-1C schematically illustrate one example of a method of locally pre-processing a CBCT scan in preparation for fusing the CBCT scan with an intraoral scan (FIG. 1A), a method of fusing the processed CBCT scan with the intraoral scan (FIG. 1B) and a method of dynamically identifying one or more defects in a treatment plan and/or modifying the treatment plan using the fused scan.

FIG. 2 illustrates one example of an overview of the method of using a CBCT scan and an intraoral scan as described herein.

FIG. 3 schematically illustrates an example of a system for fusing a CBCT scan and an intraoral scan as described herein.

FIG. 4 schematically illustrates a method of pre-processing a CBCT scan in preparation for fusing the CBCT scan with an intraoral scan.

FIG. 5 illustrates an example of a user interface for interactively displaying a final digital model of the patient's teeth comprising a fused CBCT scan and intraoral scan.

FIG. 6 shows an example of a user interface illustrating predicted root collisions in a treatment plan using a digital model of the patient's teeth comprising a fused CBCT scan and intraoral scan.

FIGS. 7A and 7B illustrate examples of a user interface showing the detection of unerupted teeth using a digital model of the patient's teeth comprising a fused CBCT scan and intraoral scan.

FIG. 8A schematically illustrates examples of fenestrations, protrusions and dehiscence.

FIG. 8B illustrates an example of a user interface showing the interactive detection of fenestration during a proposed treatment plan using a digital model of the patient's teeth comprising a fused CBCT scan and intraoral scan.

FIG. 8C illustrates examples of a user interface showing the interactive detection of fenestration during a proposed treatment plan using a digital model of the patient's teeth comprising a fused CBCT scan and intraoral scan.

FIGS. 9A and 9B show examples of a user interface configured to interactively display features detected using a digital model of the patient's teeth comprising a fused CBCT scan and intraoral scan.

FIGS. 10A and 10B show an example of a user interface for displaying a digital model of the patient's teeth comprising a fused CBCT scan and intraoral scan.

FIG. 11 schematically illustrates an example of a method of using a digital model of the patient's teeth comprising a fused CBCT scan and intraoral scan for treatment planning.

FIG. 12 shows a diagram illustrating an example of a computing environment including the CBCT scan fusion module(s), described herein.

### DETAILED DESCRIPTION

Described herein are methods and apparatuses (e.g., devices, systems, etc., including software, firmware and hardware) for identifying and visualizing problems, particularly problems arising from tooth movement, when preparing to treat a patient's teeth as part of a proposed treatment plan. These methods may advantageously include dynamically predicting and/or identifying such problems during the proposed treatment plan and allowing a user (e.g., doctor, dentist, orthodontist, etc.) to modify the proposed treatment plan and examine the effect of any modifications on these problems. These methods and apparatuses for performing them may be performed in real time. The treatment plan may be a plan for moving (e.g., aligning) a patient's teeth, removing teeth, adding a dental implant, etc.

Problems that may be identified and visualized by the methods and apparatuses described herein may include, but are not limited to: fenestration, protrusion into the sinus cavity (“protrusion”) and dehiscence. In any of these methods and apparatuses, visualizing potential problems significantly reduces the risk of unpredictable tooth movements. Unpredictable tooth movements may result when calculated movements of the teeth do not occur, e.g., due to fenestrations of the tooth into the bone tissue (at any stage of treatment). Currently there is no reliable and rapid way to predict whether bone tissue will grow at a certain speed when the tooth moves to a part of the bone or if such movement will occur. Protrusion of the tooth into the sinus can cause problems with the upper respiratory tract, so it is very important to try to prevent such movements when planning treatment. The methods and apparatuses described herein may use an improved fused digital model of a patient's dentition that advantageously includes high resolution information about both tooth crowns, e.g., from an intraoral scan of the patient's teeth, as well as cone beam computed tomography (CBCT) data on tooth roots and jaw bones. In particular, the methods and apparatuses described herein include techniques for generating a fused digital model of a patient's dentition that avoids longstanding problems when handing the file size (particularly of the CBCT scan) and compatibility between CBCT and intraoral scans.

The methods and apparatuses may detect fenestrations, protrusion and dehiscence as part of an interactive user interface. Dehiscence can be detected and visualized early in a treatment plan. For visualization, these methods and apparatuses may spline bone eruptions to 2 parts: legal and illegal, and may mark illegal eruptions for the upper and lower jaws, which may fill off the part of the root of the tooth protruding from the jawbone close to the crown. Fenestrations may be detected by identifying regions of the tooth and/or jaw geometry below the crown of the tooth. Protrusions may be predicted by determining a plane horizontal to the tooth root and identifying areas of the jawbone that protrude from the bone tissue. The methods and apparatuses described herein may automatically (or semi-automatically, e.g., with prescribed feedback from a user) pre-process a CBCT scan and fuse the pre-processed CBCT scan with an intraoral scan. The CBCT scan may be preprocessed (including local preprocessed, e.g., local to a user) to ensure that it is compatible for fusion with an intraoral scan and so that the fusion process may be performed at a remote site (e.g., by a processor that is different from the user's local processor(s)). Fusion of the CBCT scan and the intraoral scan may include matching the segmented crowns of the CBCT scan with the intra oral scan in 3D space. Once the crowns are matching, the root surfaces from the CBCT data may be stitched to the crown surface from intraoral data for each tooth to generate the fused digital model. The fused digital model may be used for treatment planning and the user may visually see the actual roots for the teeth as part of the treatment plan. The methods and apparatuses described herein may include workflows for multimodal treatment planning processes in which the fused digital model (including both CBCT data and intraoral scanning data).

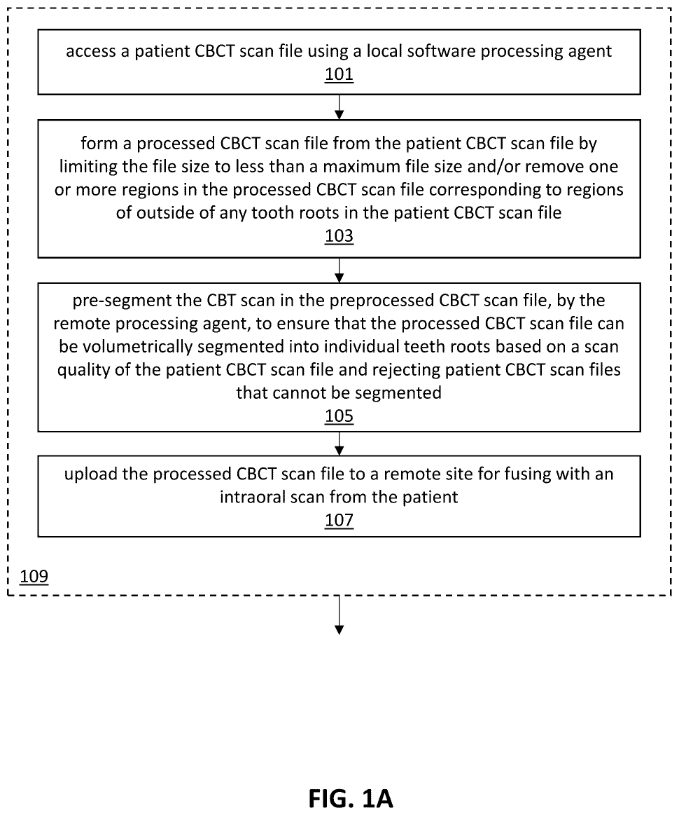

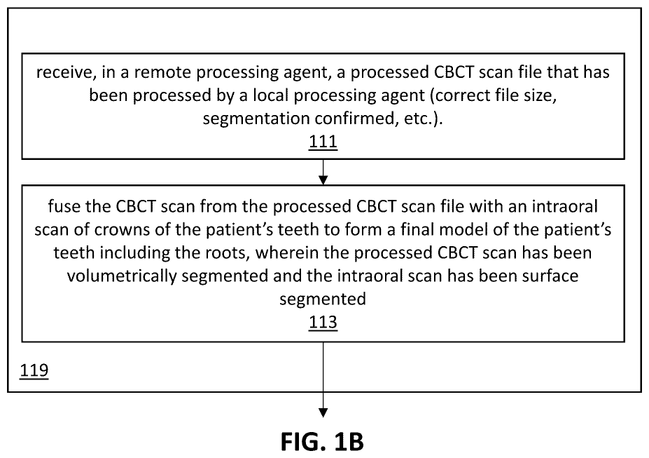

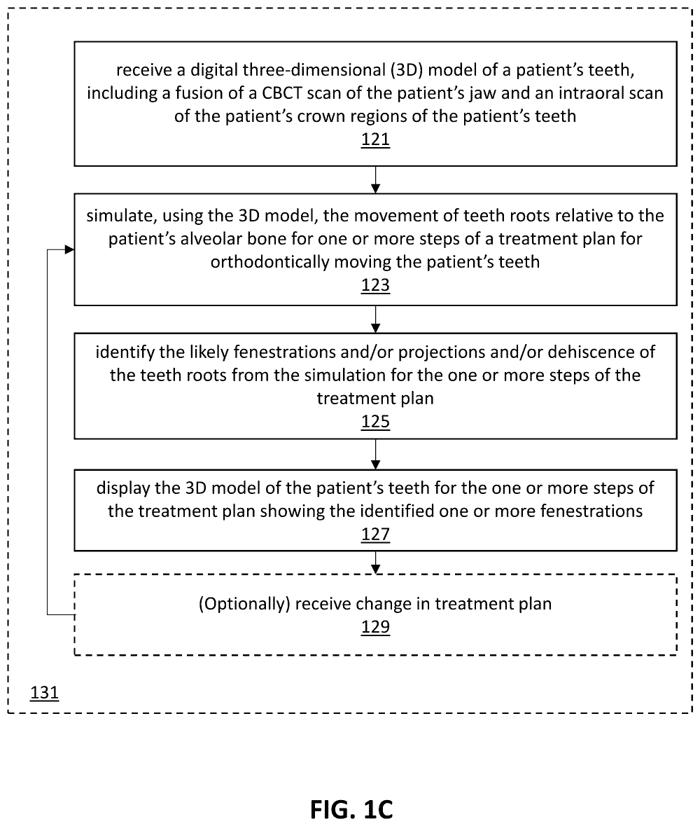

FIGS. 1A-1C collectively illustrate one example of a method for generating a fused digital 3D model of a patient's dentition including both CBCT and intraoral scan data, and interactively predicting the presence and location of problems (such as fenestrations, protrusion and/or dehiscence) arising from a proposed treatment plan for the patient's teeth. In addition, each of FIGS. 1A, 1B and 1C individually describe methods for pre-processing a CBCT scan for transmission and fusion, fusing the preprocessed CBCT scan with an intraoral scan of the patient's teeth, and interactively modifying a proposed treatment plan based on the predicted problems, respectively.

For example, FIG. 1A illustrates one example of a method of preprocessing a patient's CBCT scan in to prepare it for fusing with a second scan, such as an intraoral scan. As used herein an intraoral scan may be a surface scan, e.g., using white light or other frequencies (e.g., florescence, discrete visible light frequencies, infrared, etc.). In particular, these methods may be used for fusing a digital model of the surface of the patient's teeth taken with an intraoral scanner, which may be at relatively high resolution. The method illustrated in FIG. 1A may be for preprocessing one or more CBCT scan, and may be performed by itself or as part of a workflow for generating and/or modifying a patient treatment plan.

In FIG. 1A, the user may first reference a CBCT scan for a patient, e.g., as part of a request to generate a digital model of the patient's dentition, and/or as part of a request to generate or modify a treatment plan. Optionally, the user may request a CBCT scan be performed or may perform the CBCT scan. The CBCT scan may then be accessed as a CBCT scan file 101. The CBCT scan may be accessed from a first (e.g., local) processor using a first (e.g., local) processing agent. The first processing agent may be local to the user. The CBCT scan may be accessed from a library of patient CBCT scans and may be associated with the patient. In some cases multiple patient CBCT scans may be accessed and one or more of them selected or chosen. For example, if multiple CBCT scans are available for the same patient, the user may select one, or the local software processing agent may select one automatically (e.g., if a particular CBCT scan is rejected, it may be marked in or by the local software processing agent and another patient CBCT scan may be chosen, until an appropriate (or “best”) CBCT scan is selected.

The patient CBCT scan is then preprocessed by the local software processing agent 103. As used herein a local software processing agent may be a software agent, such as a program running on a local processor (e.g., computer, smartphone, tablet, etc.) or it may be locally accessed but distributed with a remote processor (e.g., including one or more web-accessed or cloud components). Preprocessing of the CBCT scan may be performed to normalize the size of the CBCT scan, including eliminating CBCT scan files that are too large or too small in (e.g., in one example, greater than 3 Gigabytes, less than 6 Megabytes). The local software processing agent may adjust the size of the file, including removing one or more layers and or removing (or simplifying) regions that are outside of the bone (e.g., roots, crowns and jaws) regions. For example, the local software processing agent may automatically or manually delete one or more layers of the CBCT scan from the CBCT scan file and/or may determine that one or more layers is missing. In some cases CBCT scan files that are too small, e.g., less than 6 MB, are likely to be incomplete scans.

In any of these methods and apparatuses the local software processing agent may determine the dimensions of the scanned jaw as part of the preprocessing step and may confirm that the jaw height is greater than a minimum threshold (e.g., 50 mm). For example, the local software processing agent may presume that scans with a height of less than this minimum threshold will not include all of the root apexes and therefore are likely to be incomplete. Any of the local software processing agents described herein may alternatively or additionally confirm that the slice thickness value (which may be, e.g., in the metadata associated with the CBCT scan files) is equal to and/or less than a minimum slide thickness attribute value (e.g., 0.8, 0.7, 0.6, 0.5, 0.3, 0.15, etc.). The slice thickness attribute is one example of a parameter indicative of the resolution of the CBCT scan.

In some examples the local software processing agent may examine the patient CBCT scan file to confirm that the number of layers is correct. For example, the method may determine if more than one layer of the scan is missing or corrupted. In some examples the local software processing agent may determine if the missing layers include the bone or tooth (e.g., crown, root, etc.), which may impact the necessary digital model data.

The local software processing agent may alternatively or additionally confirm that the user has selected the correct CBCT scan file(s) when submitting a case. For example, the local software processing agent may confirm that the CBCT scan has the correct extension (DCM) and/or may correct any identified errors. The local software processing agent may also or alternatively confirm that only a single CBCT scan file has been submitted as in some case the user may submit multiple scans for the same patient. In any of these methods and apparatuses the local software processing agent may identify (and prevent transmission of) non-CBCT files.

In some examples the local software processing agent may examine the quality of the CBCT scan within the CBCT scan file and may reject and/or adjust the CBCT scan file accordingly. For example, the methods and apparatuses described herein may determine the resolution of the CBCT scan file and to determine that the resolution is within an acceptable range (e.g., above some minimum resolution).

The preprocessing of the CBCT scan file by the local software processing agent is particularly important because it may help prevent problems when transferrin the CBCT scan to the remote software processing agent for fusing with the second (e.g., intraoral or surface) scan, which may lead to the treatment plan being rejected and/or may significantly delay the process. Such problems may otherwise result in the user having to re-submit the CBCT case causing delays for the patient and the treatment plan process, and may require the patient to make a new appointment and re-scan (e.g., for a new CBCT scan) resulting in an unnecessary additional dose of radiation. These issues may be prevented by preprocessing 109 as described herein, including in FIG. 1A.

In any of the methods described herein, the preprocessing step, which may be performed by the local software processing agent may confirm and/or prepare the patient CBCT scan file for segmentation. This process may be referred to as pre-segmenting the CBCT scan. Pre-segmenting the CBT scan may ensure that the output of the preprocessing, e.g., the processed CBCT scan file, can be volumetrically segmented into individual teeth roots based on a scan quality of the CBCT scan 105. Preprocessing may also result in rejecting CBCT scan files that cannot be segmented.

In some cases pre-segmenting may include applying one or more sets of rules to determine if the CBCT scan file may be segmented. For example, these method may determine if the CBCT can file may be segmented by applying a rapid partial segmentation of the CBCT scan file to identify any issues with segmentation, and particularly volumetric segmentation based on the scan file size, resolution and completeness. In some examples the CPCT scan may be segmented as part of the preprocessing step and the processed CBCT scan file may include the segmented scan file. Thus, in some examples the processed CBCT scan file may be preprocessed and segmented completely or partially by the local processing agent.

As mentioned, the preprocessing by the local processing agent may include rejecting and/or modifying the CBCT scan file, including reviewing the data and/or metadata within the CBCT scan file. Once the CBCT scan file has been preprocessed, and has not been rejected by the local processing agent, the preprocessed CBCT scan file may be uploaded as a processed CBCT scan file to a remote site for fusing with an intraoral scan from the patient 107. Thus, in FIG. 1A the output of the methods shown may be a preprocessed CBCT scan file. If the methods (or an apparatus performing this method) rejects the CBCT scan file (e.g., because of the resolution, blurriness, size, ability to be segmented, etc.) the methods may or apparatus may repeat this process, e.g., selecting another CBCT scan file, and preprocessing this next scan file; this process may be iteratively repeated until a preprocessed scan file that can be fused with an intraoral scan is passed.

Preprocessing may include the use of one or more trained machine learning agents to perform all or some of these steps. For example, a trained machine learning agent may be used to confirm that the CBCT scan may be segmented. The machine learning agent may be trained on a dataset including CBCT scans that are or are not able to be segmented (including an indicator of that the file is or is not able to be segmented). In some examples, the apparatus or method may include a trained machine learning agent that is trained to determine if one or more visual quality parameters (e.g., blurriness/crispness, resolution, etc.) is sufficient or is not sufficient.

FIG. 1B illustrates an example of a method of fusing the preprocessed CBCT scan with a second scan, such as an intraoral scan. For example, in FIG. 1B the first step include receiving the preprocessed CBCT scan file 111, which may be pre-processed as described above. The method for fusing the CBCT scan with the intraoral scan 119 described herein may include, in some examples, segmenting the CBCT scan. Any of the fusion steps shown or described as part of the method of FIG. 1B may be performed by a remote processing agent that is separate from the local processing agent. The remote processing agent may receive and store the processed CBCT scan that has been approved for fusion with the intraoral scan.

In FIG. 1B, the method of fusing the CBCT scan with the second (e.g., intraoral) scan may include receiving and/or storing a digital intraoral scan or a 3D model derived from the intraoral scan. This intraoral scan may be segmented by the remote processing agent, or it may be segmented separately, including prior to being received. Segmentation of the intraoral scan data may include segmenting of the 3D surface of the teeth. In FIG. 1B the method 119 shown may include fusing the preprocessed and segmented CBCT scan with the segmented intraoral scan. For example, fusing the CBCT scan from the processed CBCT scan file with an intraoral scan of crowns of the patient's teeth to form a final model of the patient's teeth (including the roots), may therefor include fusing a processed CBCT scan has been volumetrically segmented, and an intraoral scan has been surface segmented 113.

Fusing may include matching the surface of the crown regions from the intraoral scan to the crown region of the CBCT scan. Either or both the processed CBCT scan and the intraoral scan may be scaled and/or rotated (either by segmented region or as a whole) prior to matching and fusing. The resulting fused 3D digital model may therefore be a digital three-dimensional (3D) model of a patient's teeth, wherein the 3D model of the patient's teeth comprises a fusion of a CBCT scan of the patient's jaw and an intraoral scan of the patient's crown regions of the patient's teeth.

The digital three-dimensional (3D) model of a patient's teeth, including a fusion of a CBCT scan of the patient's jaw and an intraoral scan of the patient's crown regions of the patient's teeth (e.g., the “fused digital 3D model”) may be used to display one or more properties of the digital model, including ectopic and/or unerupted teeth, tooth roots, tooth roots and crowns, or the bones of the jaw (e.g., alveolar bone). The fused digital 3D model may be interactively displayed, and/or may be used to design a treatment plan that may be taken into account the actual (‘true’) root shape and axes.

In some example, the fused digital 3D model may be used to predict and display one or more of fenestrations, protrusions and/or dehiscence in a treatment plan. In some examples the fused digital 3D model may be used to interactively and/or iteratively revise a treatment plan. FIG. 1C illustrates one method 131 using a fused digital 3D model (such as that received from FIGS. 1B and 1A). In FIG. 1C, the fused digital 3D model may be received by the user interface 121. In some examples the user interface may be part of the local processing agent or the same system as the local processing agent. In some examples the user interface for displaying and interacting with the fused digital 3D model may be part of a remote user interface, which may be accessible to the user through a network. In some examples the user interface is part of the remote processing agent or is in communication with the remote processing agent. The method may include simulating, using the fused digital 3D model, the movement of teeth roots relative to the patient's alveolar bone for one or more steps of a treatment plan for orthodontically moving the patient's teeth 123. Movement of the teeth roots may be simulated for each stage of a treatment plan. As part of the simulation, the method may include identifying the likely fenestrations and/or projections and/or dehiscence of the teeth roots from the simulation for the one or more steps of the treatment plan 125. For example, fenestrations may be predicted based on the relative simulated movement of the tooth roots and the surrounding bone. The fused digital 3D model of the patient's teeth for the one or more steps of the treatment plan may be displayed, showing the identified one or more fenestrations 127.

In some examples the user may, based on the displayed simulated movements and/or a predicted problems, such as one or more of fenestrations and/or projections and/or dehiscence, adjust the treatment plan, including adjusting the proposed movements and/or staging of the movements, of the teeth, adding/removing attachments, removing tooth material (reductions), or the like 129. The method may then include repeating the steps of simulating identifying and displaying the fenestrations and/or projections and/or dehiscence so that the user may iteratively adjust the treatment plan.

The fused digital 3D model may therefore enable a variety of improved method for patient treatment.

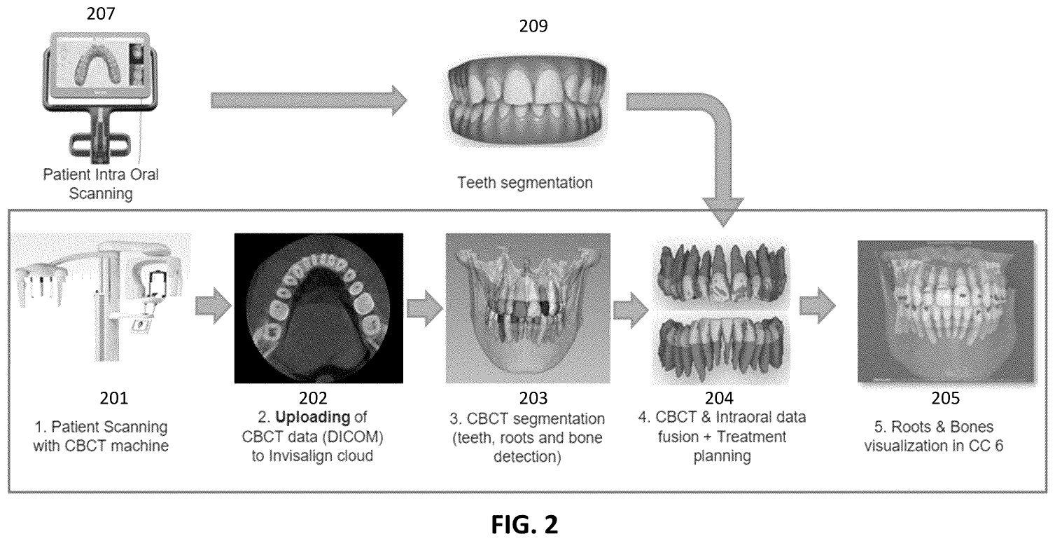

FIG. 2 shows an overview of a method of forming the fused digital 3D model as described herein. In FIG. 2, the method may include the optional step of the patient having their teeth scanned with a CBCT machine 201, and the resulting scan being uploaded (as a DIACOM) file to a database 202. These raw CBCT scan data files may be preprocessed as described above, including confirming that they may be segmented, and uploaded, with a request requiring fusion of the CBCT scan to an intraoral scan, to a remote processing agent, where they may be segmented 203 (or they may be segmented before transmission). In parallel or at different times, the patient's teeth may be scanned using an intraoral scanner 207 and the resulting scan of the patient's teeth may be segmented 209. The remote processing agent may then fuse the segmented CBCT scan 204, which may be segmented via a volume segmentation technique, with the segmented intraoral (surface) scan, which may be segmented with a surface segmentation technique. The resulting fused digital 3D model may then be presented to the user as part of a user interface 205 allowing selective visualization of the crowns, tooth roots, bone, gingiva, etc.

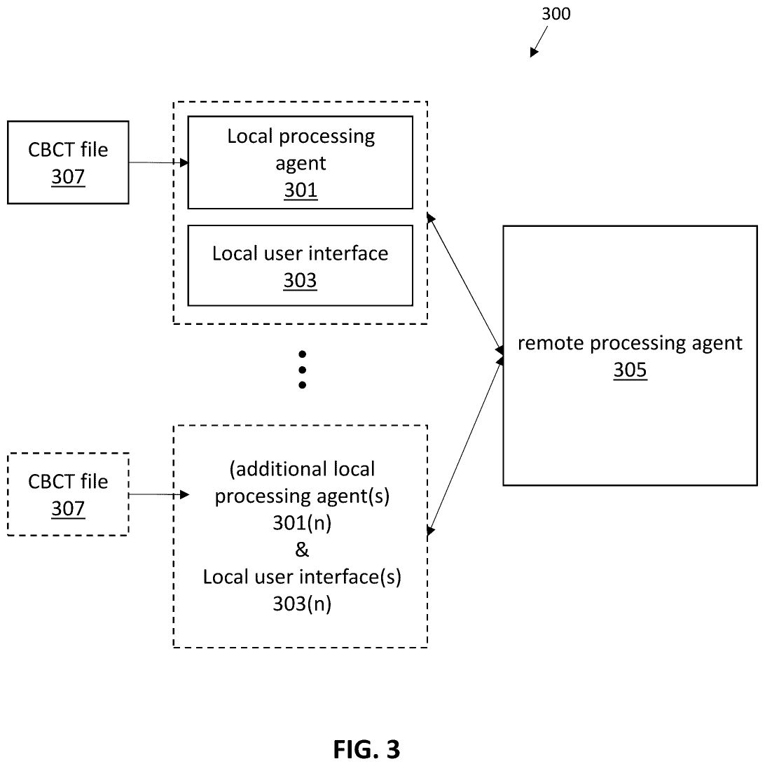

FIG. 3 illustrates one example of a system for performing the methods described herein, including the methods illustrated in FIGS. 1A, 1B and 2. In FIG. 3, the apparatus 300 includes a local processing agent 301 which may include or may be separate from a local user interface 303. The local processing agent may be configured to receive and/or access one or more (e.g., a library of) CBCT files 307. The local processing agent may include software, hardware and/or firmware and may be configured to perform any of the pre-processing steps described above. In some case multiple local agents 301(n) and/or local user interfaces 303(n) may be included as part of the system and each may access the stored CBCT files 307. The local processing agent and/or local user interface may also be in communication (including wireless communication) with a remote processing agent 305 that is configured to perform any of the steps described in FIG. 1B, including forming the fused digital 3D model. In some cases the remote processing agent 305 may be configured to perform most of the steps of FIGS. 1C and/or may output and receive input from the one or more local user interface(s) 303, 303(n).

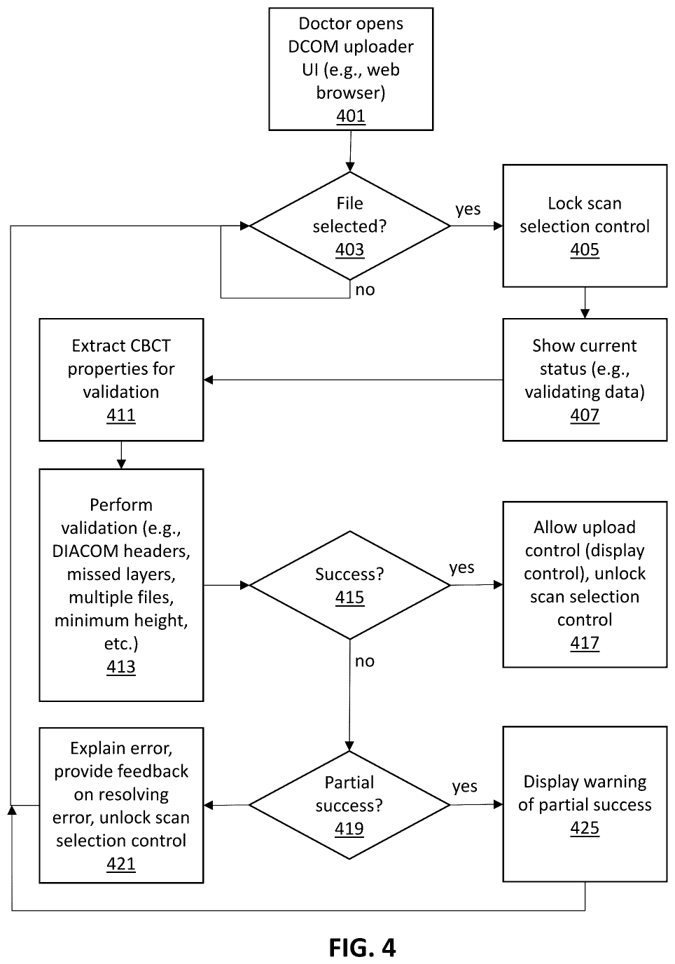

As mentioned, the method described herein may include methods of preprocessing the raw CBCT scan data (e.g., locally) so that it may be used by a (e.g., remote) processing agent to form the fused digital 3D model, and optionally to display one or more features of the fused digital 3D model and/or predict flaws (fenestrations, etc.) of a treatment plan. FIG. 4, like FIG. 1B illustrates a method of preprocessing a CBCT scan to form a processed CBCT scan. In FIG. 4, the user may first select the one or more CBCT scan data files 401. For example, this may be done in the local processing agent, local to the user, and may include using a browser (e.g., web browser) to select between one or more files. Once the file is selected 403, the local processing agent may lock the selection control, e.g. to prevent multiple files from being selected 405, and may begin preprocessing of the selected CBCT scan file. The local processing agent may show the current status of the processing agent to the user (e.g., “validating CBCT scan data”) 407 on a user interface (which may be the local user interface or a different user interface). The method may include extracting one or more properties of raw CBCT scan for analysis during preprocessing 411. The extracted properties may be extracted from the images themselves and/or from metadata (header data) in the file. The method or system may then perform the validation steps described above, such as (but not limited to) confirming that the CBCT scan data file may be segmented, adjusting the layers, adjusting the file size, confirming that no layer are missing, etc. 413. In some case a trained neural network may be used to determine one or more of these validation steps. Any of these methods may then include presenting one or more explanations of any errors or problems with the original/raw CBCT scan data. The system may then determine if (due, e.g., to any of the validation steps) if the processed CBCT scan data can be successfully used 415; if so, then the resulting preprocessed CBCT scan data file may be uploaded (or permitted to be uploaded) and the scan selection control may be unlocked 417. If not, then the system or method may determine if the validation was a partial success 419, in which chase the method or system may display a warning indication this and instruction the user how to proceed 425. Alternatively the system or method may determine that the validation stage of preprocessing was a failure, requiring feedback to resolve one or more errors (or granting approval to automatically resolve) 421, and the process may repeat to allow the user to select another file (in the case of failure) or adjust the current CMCT file (as in the partial success.

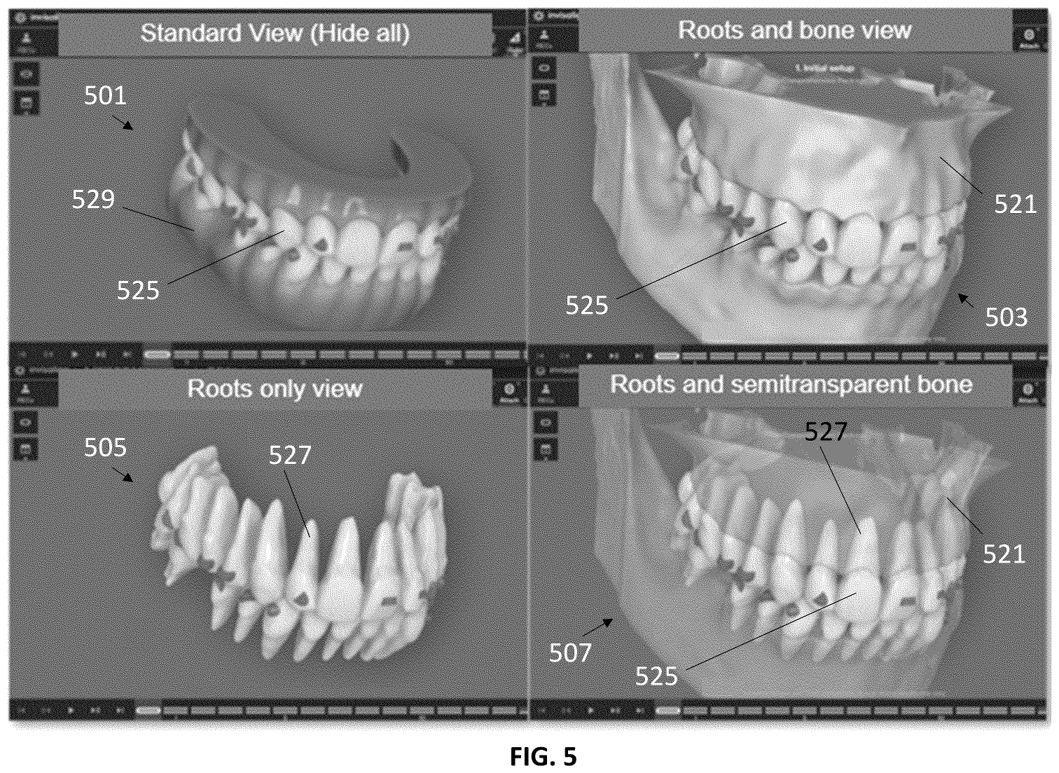

FIGS. 5-10 illustrate examples of user interfaces and the use of the fused digital 3D models described herein. For example, FIG. 5 illustrates one example of a user interface allowing display of different components (e.g., different segments) of the fused digital 3D model. In FIG. 5 (upper left) the fused digital 3D model 501 is manipulated by the user interface to receive control information through the user interface showing a standard view of the tooth crowns 525 and gingiva 529. The bottom of the user interface in this example includes selectable stage indicators allowing the user to select between different treatment plan stages. In another view 503 the user may select to show tooth crowns 525 and bone 521 (as shown in FIG. 5 upper right). In any of these views the user may rotate or translate the upper and/or lower jaw. FIG. 5 bottom left illustrates another view 505 of the user interface, showing just the roots 527 and tooth crowns. Finally FIG. 5 bottom right shows an example of a view 507 including tooth roots 527, crowns 525 and a semitransparent view of the bone 521. In operation the user may toggle between any of these views.

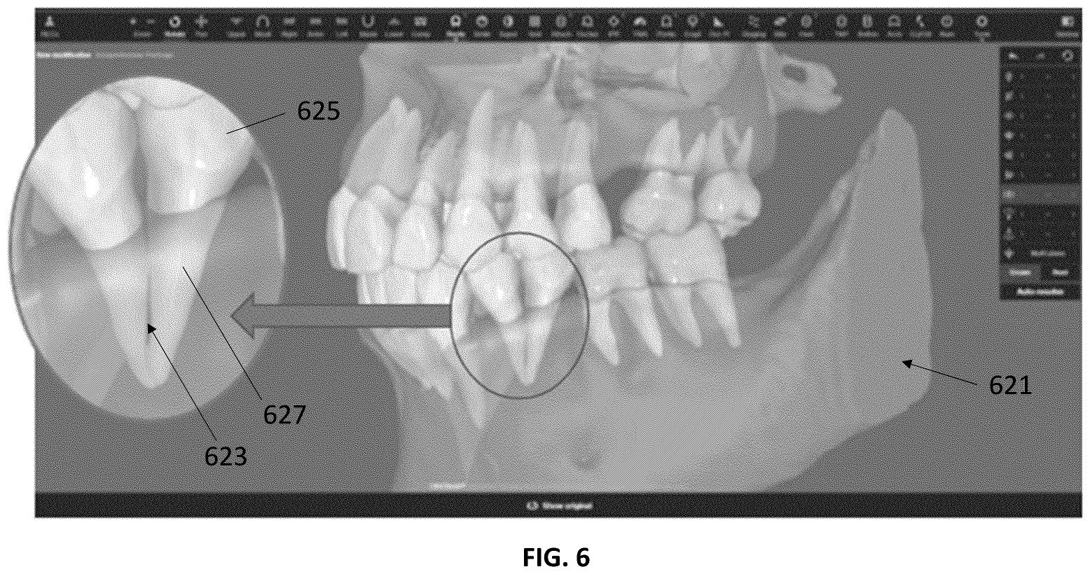

Other examples of user interface options are shown in FIG. 6. In any of these examples the methods described herein may be used to determine collisions of one or more tooth roots when generating and/or analyzing a treatment plan. For example FIG. 6 illustrates an example of a collision 623 (highlighted in the enlarged region) between a pair of tooth roots 627 during at least one treatment stage. The fused digital 3D model includes a bone region 621, crown regions 625, and the dental roots 627. The user interface in this example may automatically or semi-automatically examine the treatment plan when using the fused digital 3D model and may display an identified issue such as a collision 623, as shown. In some cases the user interface may allow the user to toggle to showing the roots and/or optionally showing collisions (after detecting collisions using the fused digital 3D model) and/or showing other features.

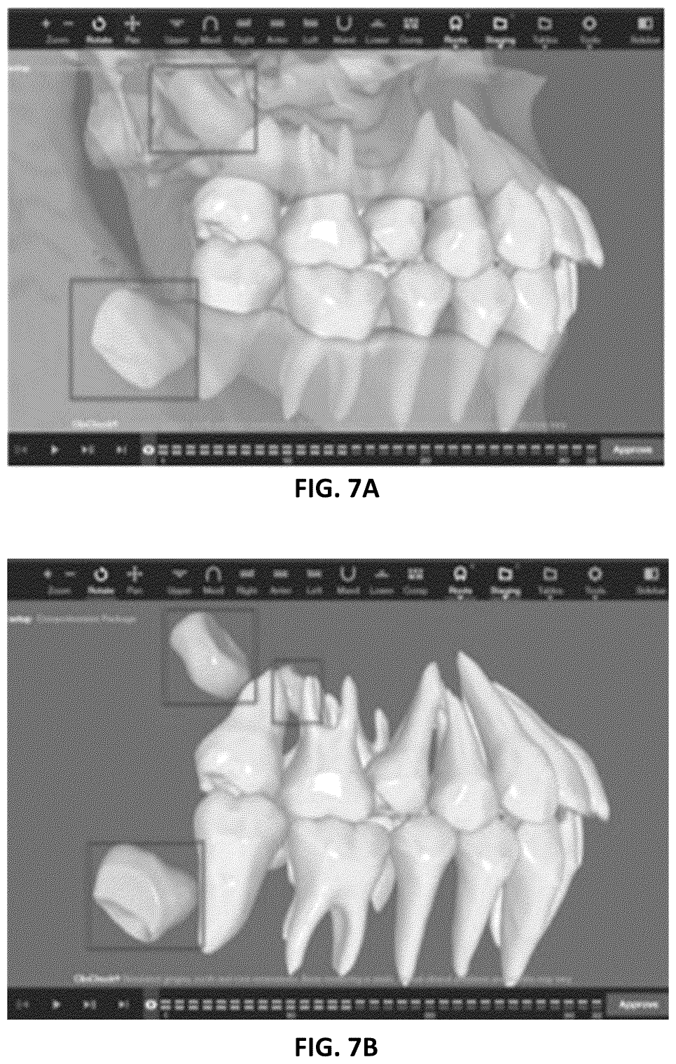

For example, FIGS. 7A-7B illustrate the use of the user interface to visualize ectopic and/or unerupted teeth. In FIG. 7A, the user interface is displaying the teeth crows and roots with the bone region around the teeth being shown as semi-transparent, revealing a pair of unerupted, ectopic teeth that may be compensated for (or removed) during the treatment. FIG. 7B shows the same view of the user interface as shown in FIG. 7A, but with the surrounding bone region made fully transparent.

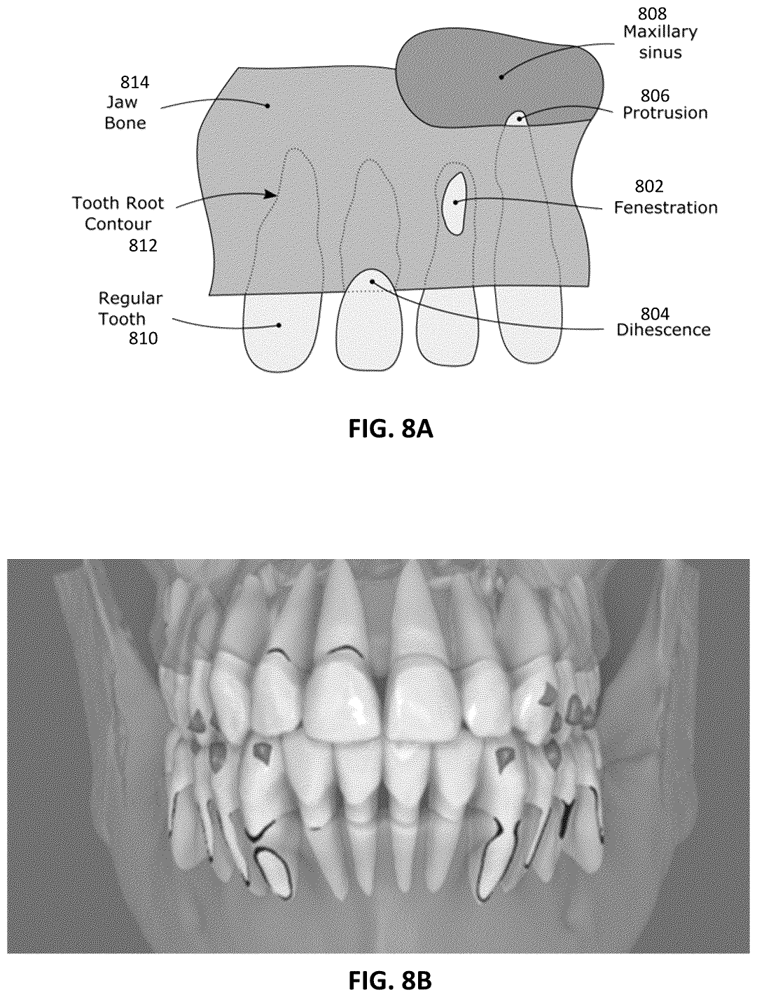

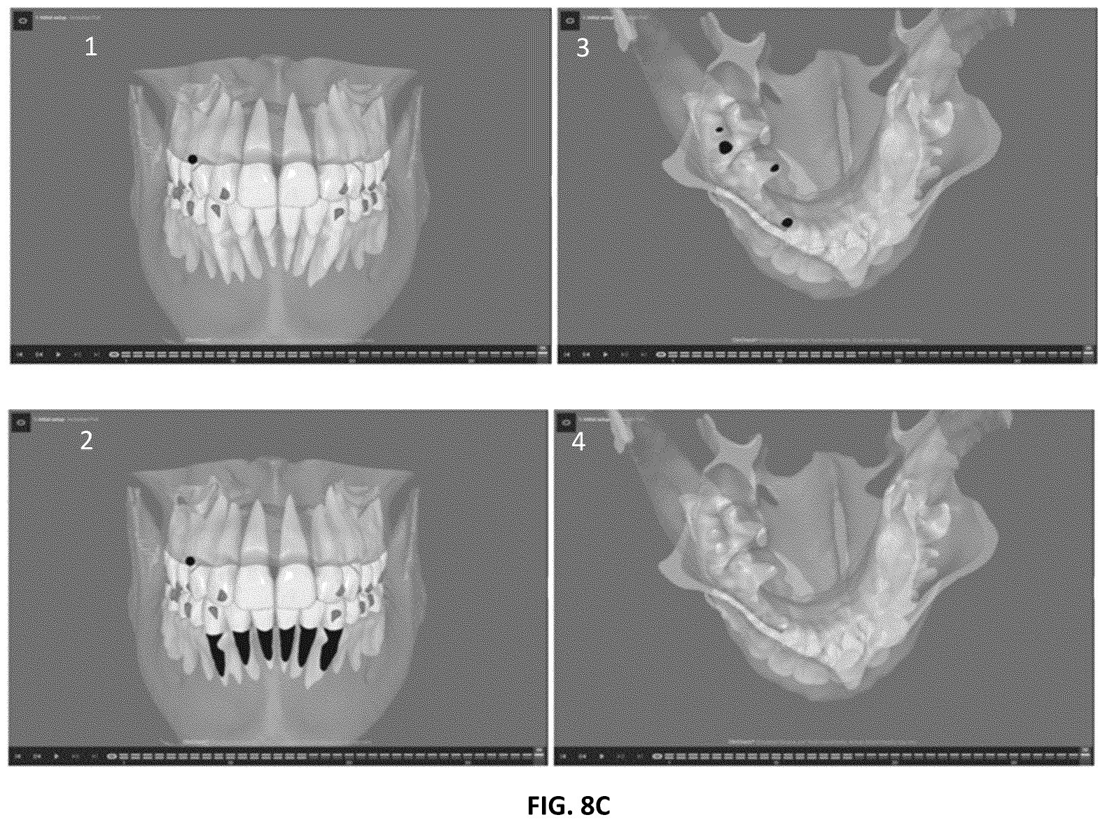

In general, these user interfaces may be used with the fused digital 3D model to display one or more predicted flaws, as illustrated in FIG. 8A, such as fenestrations 802, dehiscence 804, protrusions 806 into the sinus region 808. The user interface may also be configured to show these flaw in the context of the regular teeth 810, tooth roots 812 and/or jaw bone 814. FIG. 8B shows an example of a user interface using the fused digital 3D model to predict likely fenestrations, dehiscence, and/or protrusions from a stage of a treatment plan. The user interface may include one or more user-selectable controls to toggle between these different display options (e.g., showing one or more of predicted fenestrations, dehiscence, and/or protrusions, showing crown regions and/or roots and/or bone (as solid or translucent, etc.)). For example, the user interface may include a “show fenestrations” control input that outlines potential virtual fenestrations in the selected treatment plan stage of the fused digital 3D model.

FIGS. 8C1-8C4 illustrate user interface image showing the fused digital 3D model with the “show fenestrations” control input (e.g., toggle) off (FIG. 8C1, 8C4) and on (FIGS. 8C2, 8C3), respectively. In FIGS. 8C2 and 8C3 the front and top views of the fused digital 3D model show possible fenestrations indicated by the dark coloring.

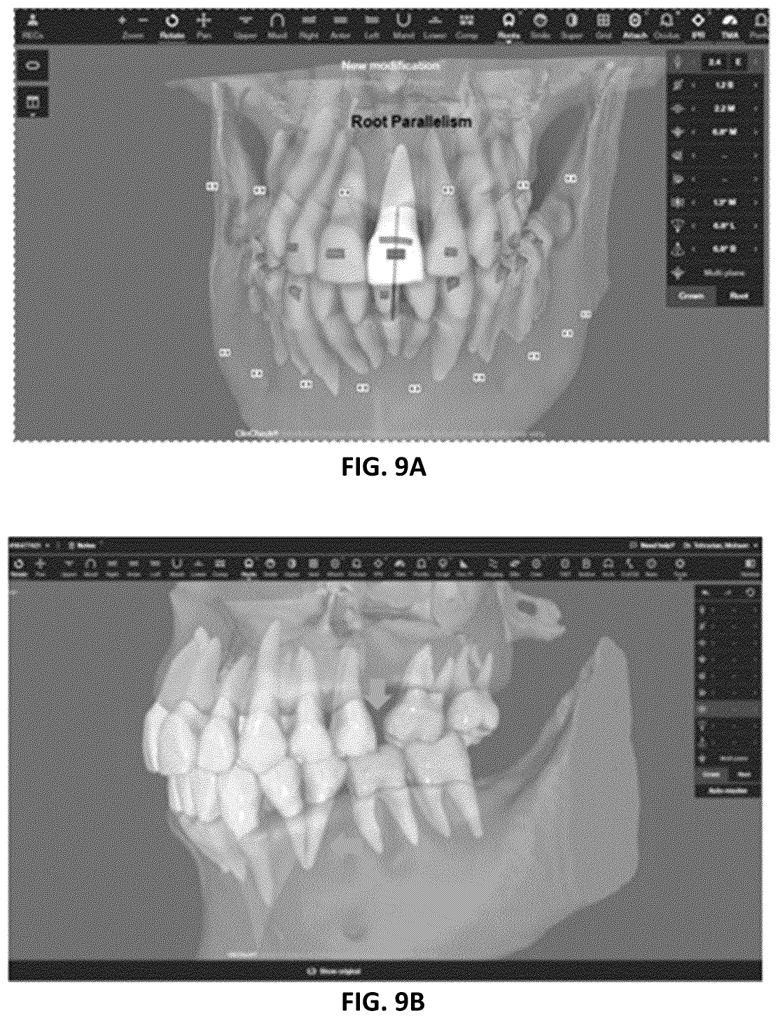

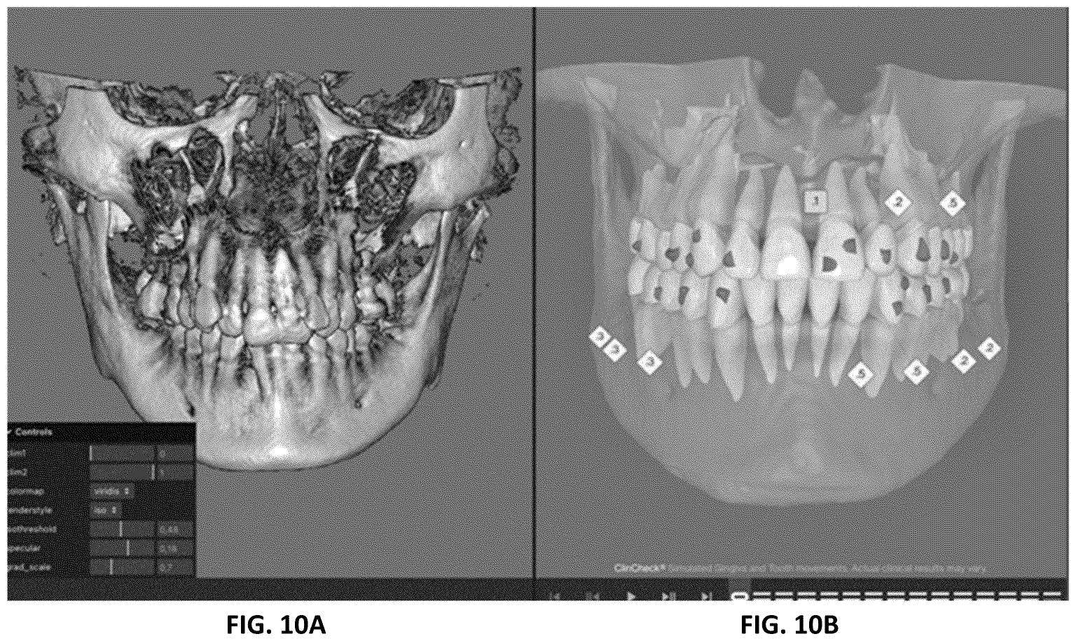

In general the user interface may display one or more features of the fused digital 3D model, as shown in FIGS. 9A-9B. For example the user interface may toggle between showing or hiding tooth roots, bone (e.g. alveolar bone/jaw bone), gingiva, etc. Individual teeth may be displayed and/or manipulated, and individual treatment stages may be displayed. For example, the user may select one or more teeth and may move it using controls on the user interface either in x, y, z or in rotation/tilt. These user-input changes to the treatment plan may modify the treatment plan and may recalculate the predicted tooth collisions/root collisions, fenestrations, etc. These treatment plans may be useful for treating a broad range of malocclusions including surgical, restorative, expansion/extraction, etc. In any of these examples the user input may also or alternatively include the display of CBCT scan (see, e.g., FIG. 10A) in addition to the display of teeth (FIG. 10B) as shown in FIGS. 5-9B, above.

As described above, traditional treatment planning for dental procedures such as dental alignment did not incorporate CBCT scans into the treatment planning process and into the generation of one or more (e.g., a series) of dental appliance to perform the treatment plan. Doctors typically proceed without the benefit of accurate tooth root information as may be acquired by CBCT scan data when it came to the root movements of a typical treatment plan and were unable to plan for roots as they could not “see” their movements throughout the treatment. The methods and apparatuses (e.g., software, hardware, firmware, etc.) may, for the first time, allow doctors to accurately visualize and plan a treatment plans with roots in mind.

Additionally, these methods and apparatuses may provide a treatment plan either automatically or semi-automatically through the data fusing processes (e.g., between the CBCT scan and intraoral scan) as described herein. This may be achieved by matching the segmented crowns of the CBCT scan with the intra oral scan in 3D space. Once the crowns are matched the root surfaces from CBCT data and crown surface from intraoral data for each tooth may be stitched together and visually shown to a technician and/or designers in a technician/designer-facing and/or clinician-facing software user interface. The technician/designer may complete the treatment plan with roots in mind. This new treatment plan with accurate, actual data on the roots may then be posted to a clinician-facing user interface where the doctor can visually see the real roots in the proposed plan and make modifications/edits.

In general these methods and apparatuses for combining CBCT scan data information with intraoral scan data may be implemented as one or more modules, including a CBCT scan fusion engine having one or more modules (e.g., preprocessing module, file reduction module, etc.). Any of these apparatuses may include one or more processors for executing the methods described herein. A processor may include hardware that runs the computer program code. Specifically, the term ‘processor’ may include a controller and may encompass not only computers having different architectures such as single/multi-processor architectures and sequential (Von Neumann)/parallel architectures but also specialized circuits such as field-programmable gate arrays (FPGA), application specific circuits (ASIC), signal processing devices and other devices.

A CBCT fusion engine may include one or more modules (e.g., a patient CBCT scan file input module, a processed CBCT scan file generating module, a processed CBCT scan truncation module, a CBCT scan pre-segmentation module, a CBCT scan file validation module, and an intraoral scan/CBCT scan stitching module) and one or more datastores (e.g., an intraoral scan data store). In some examples the CBCT fusion engine include a local CBCT scan processing engine and a remote CBCT scan processing engine. For example the local CBCT scan processing engine may include the patient CBCT scan file input module, a processed CBCT scan file generating module, a processed CBCT scan truncation module, a CBCT scan pre-segmentation module, and optionally the CBCT scan file validation module. The remote CBCT scan processing engine may include the intraoral scan/CBCT scan stitching module. Alternatively, all of these modules (e.g., the entire CBCT fusion engine, may be local, or may be remote.

The CBCT fusion engines described herein may be performed by one or more processors. As used herein, an engine includes one or more processors or a portion thereof: A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors, or a first engine and a second engine can share one or more processors with one another or other engines. As mentioned, depending upon implementation-specific or other considerations, an engine can be centralized, or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices. In some cases it may be particularly beneficial for some or all of these engines to remain local, rather than cloud-based, as indicated above.

As used herein, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

The patient CBCT scan file input module may be configured to receive a patient CBCT scan, e.g., from a CBCT scan datastore, from a remote location, or as part of a CBCT scanner. The patient CBCT scan file input module may receive and pass on the patient CBCT scan file to the processed CBCT scan file generating module. The processed CBCT scan file generating module may build a processed CBCT scan file (including a modified CBCT scan taken from the received patient CBCT scan file). The processed CBCT scan file generating module may adjust the file size of the processed CBCT scan file as described above. For example, the processed CBCT scan file generating module may truncate portions of the CBCT scan derived from the received patient CBCT scan. This may be performed by a processed CBCT scan truncation module that may be called by the processed CBCT scan file generating module or may be included as part of the processed CBCT scan file generating module and may reduce the file size as discussed above, e.g., removing portions of the scan that are outside of the tooth root or other regions. The processed CBCT scan file generating module and/or the CBCT scan truncation module may include a trained machine learning agent to perform all or a portion of the truncation and or other steps for generating the processed CBCT scan file. The processed CBCT scan file generating module may also call and/or include a CBCT scan pre-segmentation module that may pre-segment the CBCT scan of the processed CBCT scan file. The CBCT scan fusion engine may also include a CBCT scan file validation module that may confirm that the CBCT scan of the processed CBCT scan file and/or the CBCT scan of the patient CBCT scan file is appropriate for use or otherwise validate it as described above. The intraoral scan/CBCT scan stitching module may combine the intraoral scan with the CBCT scan of the processed CBCT scan file assuming that it has been validated, e.g., by the processed CBCT scan file validation module.

In general, the method and apparatuses described herein may include forming and/or fabricating one or more dental appliances from the treatment plans generated and/or modified as described herein. As used herein, generating a dental appliance may include generating a digital model of the one or more dental appliances (e.g., a series of dental appliances), including forming a digital file that may be used by a fabricator to fabricate the physical appliance to be provided to the patient. In some cases these dental appliances may be fabricated by an additive fabrication (e.g., 3D printing) technique. Thus, described herein are aligner fabrication engine(s) that may implement one or more automated agents configured to fabricate a dental appliance (e.g., aligner).

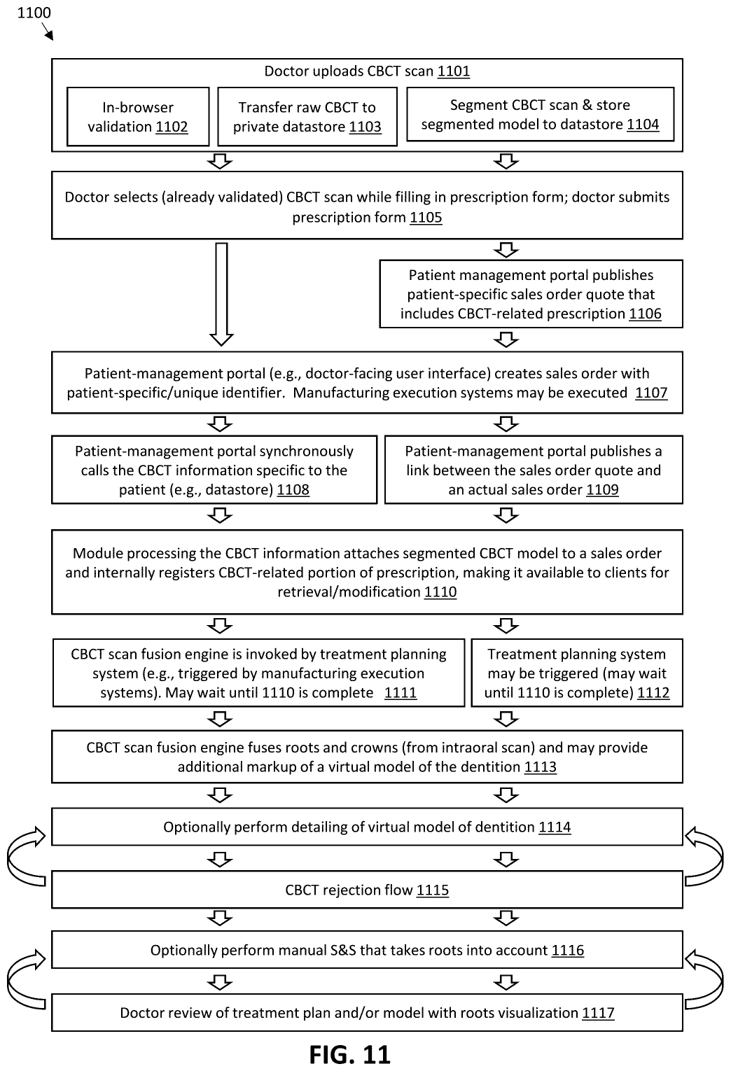

The methods for forming an improved digital model of a patient's dentition using root information from a CBCT scan and crown surface information from an intraoral scan described herein may be included as part of a workflow that improves dental and medical treatments. For example, FIG. 11 illustrates one example of a process flow 1100 that integrates the use of a CBCT scan and fusion of the CBCT scan with an intraoral scan of a patient's teeth for treatment planning as described herein. The first part of the process shown in FIG. 11 (e.g., steps 1101-1110) illustrate alternative variations for pre-processing CBCT scans and handling the CBCT scan when a user (e.g., doctor) is requesting a prescribed treatment plan, for example, for a series of dental aligners. The doctor may select the CBCT scan and/or may generate a request for a treatment plan including the CBCT scan. The method may preprocess the scan (e.g., keep/reject, modify, confirm properties), and may then fuse the CBCT scan with an intraoral scan (Steps 1111-1115). The treatment planning may then be performed using the resulting fused digital 3D model (steps 1116-1117).

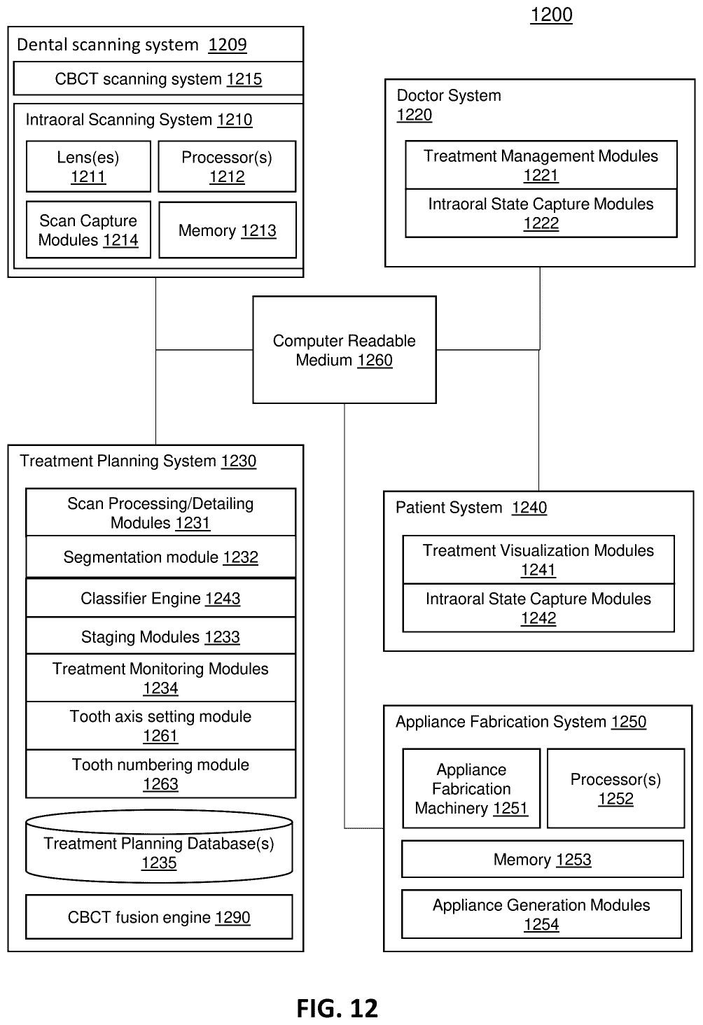

For example, the doctor may initially upload a CBCT scan into a datastore or system for generating a treatment plan 1101 (see, e.g., FIG. 12, below). Uploading may be performed as part of the CBCT scanner, or separately, including as part of a user interface that may be part of a web browser. Prior to uploading the scan, or as part of the uploading process, the system may perform in-browser validation of the CBCT scan 1102. This may include confirming the integrity of the scan and/or the scan file. This may include verifying the association with a particular patient, e.g., linking to an existing patient file. This may optionally include formatting or other pre-processing steps for the patient CBCT scan. In some cases uploading may include transferring the patient CBCT scan (e.g., the raw CBCT scan) to a database/datastore 1103. In some cases the datastore may be local and may be accessible by the user (e.g., doctor) or it may be remote (e.g., cloud based, such as part of a commercial web service) 1103; in some cases the CBCT scan is stored in a secure cloud based datastore. In some cases, as described above, the patient CBCT scan may be preprocessed to generate a processed CBCT scan file from the patient CBCT scan file that has been preprocessed as described herein, and this processed CBCT scan file may be stored 1104 instead of or in addition to the ‘raw’ patent CBCT scan file. For example, the size of the CBCT scan taken from the patient CBCT scan file may be reduced/modified, as described herein.

Once the CBCT scan file(s) is/are uploaded and associated with the patient, the doctor may select the CBCT scan when filling in a patient prescription form for performing a dental treatment and may submit the prescription form 1105. This may be done through part of a doctor system/sub-system (as described in reference to FIG. 12, below), and may include a doctor-facing user interface that may allow the doctor to select to use the CBCT scan as part of the treatment plan preparation. As part of this step, the user interface, which may be or be part of a patient management portal, may provide a patient-specific sales order quote that includes CBCT-related prescription 1106. In some cases the patient management portal may include or be part of a patient system, as described in greater detail in FIG. 12, below. The patient-management portal (e.g., doctor-facing user interface) may create a sales order with the patient-specific (e.g., unique) identifier that is correlated with a particular user and/or doctor. This process may also engage the treatment planning system and/or an appliance fabrication system/subsystem (e.g., the manufacturing execution systems) 1107. The patient management portal may synchronously access the datastore containing the CBCT information and/or any additional data specific to the patient and the scans (e.g., CBCT scan, intraoral scan, etc.) 1108, and may present (‘publish’) a link between the sales order quote and an actual sales order 1109.

The system, including a module that may coordinate the accessed CBCT information and the doctor's prescription, may attach (or link to) the pre-processed (e.g., segmented) CBCT scan data to a sales order and may internally register the CBCT-related portion of prescription, making it available to clients for retrieval/modification 1110. This may be part of the same doctor-facing subsystem (e.g., patient management portal) or may be a separate module.

The system may then perform an initial fusion of the CBCT scan and an intraoral scan. The CBCT scan fusion engine may be invoked by the treatment planning system and may be accessed or triggered by the treatment planning and/o manufacturing sub-systems 1111. This step may be performed after the client (e.g., doctor) is done modifying and/or accepting the sales order (e.g., step 1110). The process may trigger the treatment planning system 1112.

Thereafter, the CBCT scan fusion engine may fuse the tooth roots in the CBCT scan with the tooth crowns from the intraoral scan, and may otherwise process the virtual model of the patient's dentition 1113. This may include further annotating the digital model of the patient's dentition (including labeling one or more features, identifying patient data/metadata, etc.).

Optionally, the process may include additional detailing of the digital model. This detailing may be automatic, semi-automatic (e.g., technician/physician assisted and/or approved), or manual 1114. The user (e.g. physician) may the approve, reject and/or suggest additional modifications to the fused digital model as part of a CBCT rejection flow 1115, which may be a manual step. Optionally, the method may then use the digital model in further processing including treatment planning, including an automatic, semi-automatic or manual S&S that takes roots into account 1116. The user, e.g., doctor may then review the treatment plan and/or digital model(s) using roots visualization 1117.

FIG. 12 is a diagram illustrating one variation of a computing environment 1200 that may generate one or more orthodontic/dental appliances and/or treatment plans specific to a patient, and fabricate dental appliances that may accomplish the treatment plan to treat a patient, under the direction of a dental professional. The computing environment may include a CBCT fusion engine 1290 as described above. The example computing environment 1200 shown in FIG. 12 includes a dental scanning system (e.g., optionally including an intraoral scanning system 1210, and/or CBCT scanning system 1215), a doctor system 1220, a treatment planning system 1230 (e.g., technician system), a patient system 1240, an appliance fabrication system 1250, and computer-readable medium 1260. Each of these systems may be referred to equivalently as a sub-system of the overall system (e.g., computing environment). Although shown as discrete systems, some or all of these systems may be integrated and/or combined. In some variations a computing environment (dental computing system) 1200 may include just one or a subset of these systems (which may also be referred to as sub-systems of the overall system 1200). As mentioned, one or more of these systems may be combined or integrated with one or more of the other systems (sub-systems), such as, e.g., the patient system and the doctor system may be part of a remote server accessible by doctor and/or patient interfaces. The computer readable medium 1260 may divided between all or some of the systems (subsystems); for example, the treatment planning system and appliance fabrication system may be part of the same sub-system and may be on a computer readable medium 1260. Further, each of these systems may be further divided into sub-systems or components that may be physically distributed (e.g., between local and remote processors, etc.) or may be integrated.

A dental scanning system 1209 may include one or more of: an intraoral scanning system 1210, and/or CBCT scanning system 1215. The intraoral scanning system may include an intraoral scanner as well as one or more processors for processing images. For example, an intraoral scanning system 1210 can include optics 1211 (e.g., one or more lenses, filters, mirrors, etc.), processor(s) 1212, a memory 1213, and a scan capture module 1214. In general, the intraoral scanning system 1210 can capture one or more images of a patient's dentition. Use of the intraoral scanning system 1210 may be in a clinical setting (doctor's office or the like) or in a patient-selected setting (the patient's home, for example). In some cases, operations of the intraoral scanning system 1210 may be performed by an intraoral scanner, dental camera, cell phone or any other feasible device.

The optical components 1211 may include one or more lenses and optical sensors to capture reflected light, particularly from a patient's dentition. The scan capture module 1214 can include instructions (such as non-transitory computer-readable instructions) that may be stored in the memory 1213 and executed by the processor(s) 1212 to can control the capture of any number of images of the patient's dentition.

In FIG. 12 the segmentation 1232 and verification 1232 (e.g., classifier engine 1243) are shown as part of the treatment planning sub-system 1230, however in some examples some, or all, of these components may be part of (or duplicated in) the dental scanning system 1209. For example, the segmentation module 1232 may be in the intraoral scanning sub-system 1210 or another sub-system may be in the treatment planning sub-system 1230 or some other sub-system. Any of the component systems or sub-systems of the dental computing environment 1200 may access or use the segmented data. Similarly the tooth axis setting module 1261 and/or the tooth numbering module 1263 may be part of the treatment planning sub-system 1230, however in some examples some, or all, of these components may be part of (or duplicated in) the dental scanning system 1209.

The doctor system 1220 (e.g., doctor sub-system) may include a treatment management module 1221 and an intraoral state capture module 1222 that may access or use the 3D model based on the segmented data. The doctor system 1220 may provide a “doctor facing” interface to the computing environment 1200. The treatment management module 1221 can perform any operations that enable a doctor or other clinician to manage the treatment of any patient. In some examples, the treatment management module 1221 may provide a visualization and/or simulation of the patient's dentition with respect to a treatment plan. This user interface may also include display the segmentation.

The intraoral state capture module 1222 can provide images of the patient's dentition to a clinician through the doctor system 1220. The images may be captured through the dental scanning system 1209 and may also include images of a simulation of tooth movement based on a treatment plan.

In some examples, the treatment management module 1221 can enable the doctor to modify or revise a treatment plan, particularly when images provided by the intraoral state capture module 1222 indicate that the movement of the patient's teeth may not be according to the treatment plan. The doctor system 1220 may include one or more processors configured to execute any feasible non-transitory computer-readable instructions to perform any feasible operations described herein.

Alternatively or additionally, the treatment planning system 1230 may include any of the methods and apparatuses described herein, including the CBCT fusion engine 1290. Although the CBCT fusion engine 1290 is shown as part of the treatment planning system 1230, the CBCT fusion engine may be separate and may communicate directly or indirectly with the Doctor System 1220 and/or the treatment planning system 1230, or any other system or sub-system shown.

The treatment planning system 1230 may include scan processing/detailing module 1231, segmentation module 1232, classifier engine(s) 1243, staging module 1233, and treatment monitoring module 1234, and a treatment planning database(s) 1235. In general, the treatment planning system 1230 can determine a treatment plan for any feasible patient. The scan processing/detailing module 1231 can receive or obtain dental scans (such as scans from the dental scanning system 1209) and can process the scans to “clean” them by removing scan errors and, in some cases, enhancing details of the scanned image. The treatment planning system 1230 may perform segmentation. For example, a treatment planning system may include a segmentation module 1232 that can segment a dental model into separate parts including separate teeth, gums, jaw bones, and the like. In some cases, the dental models may be based on scan data from the scan processing/detailing module 1231 (and/or classifier engine 1243).

The staging module 1233 may determine different stages of a treatment plan. Each stage may correspond to a different dental aligner. The staging module 1233 may also determine the final position of the patient's teeth, in accordance with a treatment plan. Thus, the staging module 1233 can determine some or all of a patient's orthodontic treatment plan. In some examples, the staging module 1233 can simulate movement of a patient's teeth in accordance with the different stages of the patient's treatment plan.

An optional treatment monitoring module 1234 can monitor the progress of an orthodontic treatment plan. In some examples, the treatment monitoring module 1234 can provide an analysis of progress of treatment plans to a clinician. Although not shown here, the treatment planning system 1230 can include one or more processors configured to execute any feasible non-transitory computer-readable instructions to perform any feasible operations described herein.

The Tooth axis setting module 1261 and/or Tooth numbering module 1263 may be included as part of the treatment planning sub-system 1230 as discussed above, and may include the features and perform the steps described above foe either tooth numbering/setting or verification and/or for tooth axis determination, setting and/or verification.

The patient system 1240 can include a treatment visualization module 1241 and an intraoral state capture module 1242. In general, the patient system 1240 can provide a “patient facing” interface to the computing environment 1200. The treatment visualization module 1241 can enable the patient to visualize how an orthodontic treatment plan has progressed and also visualize a predicted outcome (e.g., a final position of teeth).

In some examples, the patient system 1240 can capture dentition scans for the treatment visualization module 1241 through the intraoral state capture module 1242. The intraoral state capture module can enable a patient to capture his or her own dentition through the dental scanning system 1209. Although not shown here, the patient system 1240 can include one or more processors configured to execute any feasible non-transitory computer-readable instructions to perform any feasible operations described herein.

The appliance fabrication system 1250 can include appliance fabrication machinery 1251, processor(s) 1252, memory 1253, and appliance generation module 1254. In general, the appliance fabrication system 1250 can directly or indirectly fabricate aligners to implement an orthodontic treatment plan. In some examples, the orthodontic treatment plan may be stored in the treatment planning database(s) 1235. Any of these apparatuses and methods may be configured to include the step of fabricating one or more (e.g., a series) of dental appliances using a 3D model (e.g., including using the corrected segmentation, as described herein).

The appliance fabrication machinery 1251 may include any feasible implement or apparatus that can fabricate any suitable dental aligner. The appliance generation module 1254 may include any non-transitory computer-readable instructions that, when executed by the processor(s) 1252, can direct the appliance fabrication machinery 1251 to produce one or more dental aligners. The memory 1253 may store data or instructions for use by the processor(s) 1252. In some examples, the memory 1253 may temporarily store a treatment plan, dental models, or intraoral scans.

The computer-readable medium 1260 may include some or all of the elements described herein with respect to the computing environment 1200. The computer-readable medium 1260 may include non-transitory computer-readable instructions that, when executed by a processor, can provide the functionality of any device, machine, or module described herein.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof: As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of fusing a cone-beam computed tomography (CBCT) scan with an intraoral scan, the method comprising:
receiving, in a remote processing agent, a processed CBCT scan file that has been processed by a local processing agent from a patient CBCT scan file by:
removing one or more regions in the processed CBCT scan file corresponding to one or more regions in the patient CBCT scan file corresponding to regions outside of any tooth roots and/or pre-segmenting the CBCT scan in the processed CBCT scan file by the remote processing agent to ensure that the processed CBCT scan file can be volumetrically segmented into individual teeth roots based on a scan quality of the CBCT scan and rejecting patient CBCT scan files that cannot be segmented; and
fusing the CBCT scan from the processed CBCT scan file with an intraoral scan of crowns of the patient's teeth to form a final model of the patient's teeth including the roots, wherein the processed CBCT scan has been volumetrically segmented and the intraoral scan has been surface segmented.

2. The method of claim 1, wherein receiving the processed CBCT scan file comprises receiving the processed CBCT scan file that has been processed to limit the file size to less than the maximum file size by truncating the patient CBCT scan file to remove one or more regions outside of regions containing tooth roots.

3. The method of claim 1, wherein receiving the processed CBCT scan file comprises receiving the processed CBCT scan file that has been processed to limit the file size to less than the maximum file size by truncating the patient CBCT scan file to remove one or more layers of the patient CBCT scan file.

4. The method of claim 1, wherein receiving the processed CBCT scan file comprises receiving the processed CBCT scan file that has been processed to pre-segment the CBCT scan file based on the scan quality to reject CBCT scan files that cannot be segmented based on the scan quality that are blurry and/or below a minimum resolution threshold.

5. The method of claim 1, wherein receiving the processed CBCT scan file comprises receiving the processed CBCT scan file that has been processed to pre-segment the CBCT scan file by applying a trained neural network to determine if the CBCT scan file can be segmented, wherein the trained neural network is trained on database of CBCT scans having different scan qualities.

6. The method of claim 1, wherein receiving the processed CBCT scan file comprises receiving the processed CBCT scan file that has been volumetrically segmenting by the local processing agent.

7. The method of claim 1, further comprising volumetrically segmenting the received processed CBCT scan file.

8. The method of claim 1, further comprising segmenting the intraoral scan.

9. The method of claim 1, wherein fusing comprises matching crown regions of the segmented intraoral scan with crown regions of the segmented CBCT scan and replacing the crown regions of the segmented CBCT scans with the crown regions of the segmented intraoral scanner.

10. The method of claim 1, wherein fusing comprises verifying the fusion based on degree of match between the crown regions of the segmented intraoral scan with crown regions of the segmented CBCT scan.

11. The method of claim 1, further comprising determining a long axis for each tooth of the final model using a root axis of each tooth.

12. The method of claim 1, further comprising displaying the final model of the patient's teeth in a user interface configured to allow a user to interactively display subsets of segmented regions of the final model.

13. (canceled)

14. The method of claim 1, wherein the CBCT scan file comprises a Digital Imaging and Communications in Medicine (DICOM) file.

15. A method of fusing a cone-beam computed tomography (CBCT) scan with an intraoral scan, the method comprising:
receiving in a remote processing agent a processed CBCT scan file that has been processed by a local processing agent by:
preparing the processed CBCT scan file from a CBCT scan of a patient CBCT scan file, including adjusting the processed CBCT scan file to remove one or more regions outside of any tooth roots, and by
pre-segmenting to ensure that the processed CBCT scan file can be volumetrically segmented into individual teeth roots based on a scan quality of the CBCT scan and rejecting CBCT scan files that cannot be segmented;
segmenting the processed CBCT scan file in the remote processing agent;
fusing the segmented CBCT scan with a segmented intraoral scan of crowns of the patient's teeth to form a final model of the patient's teeth including the roots.

16. A non-transitory computing device readable medium having instructions stored thereon that are executable by a processor of a remote processing agent to cause the remote processing agent to perform a method comprising:
receiving a processed CBCT scan file that has been processed by a local processing agent by:
removing one or more regions in the processed CBCT scan file corresponding to one or more regions in a patient CBCT scan file corresponding to regions outside of any tooth roots and/or pre-segmenting the CBCT scan in the processed CBCT scan file by the remote processing agent to ensure that the processed CBCT scan file can be volumetrically segmented into individual teeth roots based on a scan quality of the CBCT scan and rejecting patient CBCT scan files that cannot be segmented; and
fusing the CBCT scan from the processed CBCT scan file with an intraoral scan of crowns of the patient's teeth to form a final model of the patient's teeth including the roots, wherein the processed CBCT scan has been volumetrically segmented and the intraoral scan has been surface segmented.

17. The non-transitory computing device readable medium of claim 16, wherein preparing the patient CBCT scan file comprises limiting the file size of the patient CBCT scan file to less than a maximum file size.

18. The non-transitory computing device readable medium of claim 16, wherein receiving the processed CBCT scan file comprises receiving the processed CBCT scan file that has been processed to limit the file size to less than the maximum file size by truncating the patient CBCT scan file to remove one or more regions outside of regions containing tooth roots.

19. The non-transitory computing device readable medium of claim 16, wherein receiving the processed CBCT scan file comprises receiving the processed CBCT scan file that has been processed to limit the file size to less than the maximum file size by truncating the patient CBCT scan file to remove one or more layers of the patient CBCT scan file.

20. The non-transitory computing device readable medium of claim 16, wherein receiving the processed CBCT scan file comprises receiving the processed CBCT scan file that has been processed to pre-segment the CBCT scan file based on the scan quality to reject CBCT scan files that cannot be segmented based on the scan quality that are blurry and/or below a minimum resolution threshold.

21. The non-transitory computing device readable medium of claim 16, wherein receiving the processed CBCT scan file comprises receiving the processed CBCT scan file that has been processed to pre-segment the CBCT scan file by applying a trained neural network to determine if the CBCT scan file can be segmented, wherein the trained neural network is trained on database of CBCT scans having different scan qualities.

22.-64. (canceled)
